(19) United States
(12) Patent Application Publication
(10) Pub. No.: US 2022/0202835 A1
(43) Pub. Date: Jun. 30, 2022

(54) INJECTABLE CLORSULON COMPOSITIONS, METHODS AND USES THEREOF

(71) Applicant: Boehringer Ingelheim Animal Health USA Inc., Duluth, GA (US)

(72) Inventors: Todd PRESCOTT, Suwanee, GA (US); Izabela GALESKA, Newtown, PA (US); Marilyn C. YAMAT, Bergenfield, NJ (US); Siddhi M. DUDHAT, Jersey City, NJ (US)

(73) Assignee: Boehringer Ingelheim Animal Health USA Inc., Duluth, GA (US)

(21) Appl. No.: 17/429,707

(22) PCT Filed: Feb. 28, 2020
(86) PCT No.: PCT/US20/20251

§ 371 Date: Aug. 10, 2021

Related U.S. Application Data
(60) Provisional application No. 62/812,374, filed on Mar. 1, 2019.

(51) Int. Cl. A61K 31/63; A61K 9/00; A61K 47/10; A61K 9/08; A61K 47/22; A61K 31/277; A61K 31/7048; A61P 33/10

(52) U.S. Cl. CPC A61K 31/63; A61K 9/0019; A61K 47/10; A61P 33/10; A61K 47/22; A61K 31/277; A61K 31/7048; A61K 9/08

(57) ABSTRACT

The invention relates to injectable compositions for combating liver fluke parasites in mammals, comprising clorsulon as the active agent. The invention also provides for an improved method for eradicating and controlling liver fluke parasite infections in a mammal comprising administering the compositions of the invention to the mammal in need thereof.

### CROSS REFERENCE TO RELATED APPLICATIONS

This application claims the benefit of priority to U.S. Provisional Application No. 62/812,374, filed Mar. 1, 2019, which is incorporated herein by reference.

### FIELD OF THE INVENTION

The present invention provides injectable veterinary compositions comprising clorsulon for controlling liver flukes in mammals. The use of these compositions against liver flukes and methods for treating parasitic infections and infestations in mammals is presented herein.

### BACKGROUND OF THE INVENTION

Animals such as mammals (including humans) are often susceptible to parasite infections and infestations. These parasites may be ectoparasites, such as insects, and endoparasites such as filariae and other worms. Production animals, such as cows, pigs, sheep and goats, can be infected with one or more trematodes (also called “flukes”). Of particular concern here is genus Fasciola (i.e., liver fluke) and genus Fascioloides (i.e., deer fluke). Several exemplary species are Fasciola hepatica, Fasciola gigantica and Fascioloides magna.

Liver flukes are a particular problem because they adversely affect the health of the animal or human and can inflict significant economic loss in a domestic livestock population. It is estimated that F. hepatica poses a risk to at least 250 million sheep and 350 million cattle worldwide. Moreover, domestic animals other than sheep and cows may serve as intermediate hosts. Liver flukes can cause liver condemnation, secondary infections, reduced milk and meat production, abortion and fertility problems.

Several types of control measures for liver flukes have been introduced over the past century. First, halogenated hydrocarbons (e.g., carbon tetrachloride) were introduced for ruminants in the 1920s. Halogenated hydrocarbons had limited success and are no longer used primarily because of their adverse effects and variable efficacy. Second, halogenated phenols were administered in the late 1950s (e.g., hexachlorophene and bithionol sulfoxide) followed by the similar halogenated salicylanilides (e.g., oxyclozanide, bromoxanide). Fourth, benzimidazole carbamates (e.g., albendazole, luxabendazole) were found to have a broad anthelmintic spectrum against nematodes and adult F. hepatica. Another benzimidazole—the chlorinated methylthiobenzimidazole derivative triclabendazole—has a high success rate against F. hepatica. Fifth, bisanilino compounds introduced in the 1960s were intolerable due to toxic side effects. Finally, benzene sulfonamides (e.g., clorsulon) were studied in the 1970s. Extensively modified examples of this class demonstrate high efficacy on both adult and immature F. hepatica.

The benzimidazole anthelmintics are widely used to treat internal worm parasites. Representatives of this anthelmintic class can be found in, for example, U.S. Pat. No. 4,197,307 (discloses 6-phenyl substituted benzimidazoles useful for treating trematodes), U.S. Pat. No. 4,205,077 (discloses benzimidazole sulfides as anthelmintic agents), U.S. Pat. No. 4,336,262 (discloses a pour-on anthelmintic that is heavily substituted at the 7-position of the benzimidazole ring) and U.S. Pat. No. 4,468,390 (discloses an anthelmintic composition that is a mixture of a macrolide antibiotic and one of a benzimidazole, a salicylamide or an isoquinoline compound).

Indeed, triclabendazole is the current drug of choice against adult and immature liver flukes. Not surprisingly, however, reports of parasite resistance are increasing. For example, Mottier et al., report that a population of resistant F. hepatica (Sligo strain) may use an altered influx/efflux mechanism to selectively decrease the amount of triclabendazole and triclabendazole sulfoxide but not albendazole. See Mottier et al., J. Parasitol., 92(6), 2006, pp. 1355-1360. McConville et al., report that juvenile triclabendazole-resistant F. hepatica are somewhat susceptible to compound alpha (i.e., 5-chloro-2-methylthio-6-(1-naphthyloxy)-1H-benzimidazole) via a tubulin-independent mechanism. See McConville et al., Parasitol. Res., (2007) 100:365-377. Further, Keiser et al., report the testing of artemether and OZ78 in triclabendazole-resistant F. hepatica, although at high concentrations. For a short review of triclabendazole resistance see Brennan et al., Experimental and Molecular Pathology, 82, (2007) pp. 104-109.

The resistance to triclabendazole and lack of effective substitutes creates a pressing need in the field for alternatives that exhibit low side effects and that do not contaminate the animals as a food source. Optimal compositions should further be efficacious, have a quick onset of activity, and be safe to the animal recipients and their human owners.

In this regard, the aforementioned class of benzene sulfonamides is known to have antiparasitic activity against liver flukes. U.S. Pat. No. 4,001,406 to Mrozik discusses such activity of 1-amino-haloalkyl-4,6-benzenedisulfonamide derivatives and U.S. Pat. No. 4,062,952, also to Mrozik, focuses on 4-amino-1,3-benzenedisulfonamide derivatives.

As a member of the benzenedisulfonamide class, clorsulon has been used to combat fascioliasis. Clorsulon's reported mechanism of action is to inhibit various enzymes involved in the glycolytic process of flukes, making it difficult for the flukes to obtain energy from glucose. Consequently, the levels of ATP, the cellular fuel, are depressed and the worms die.

Typically, clorsulon is used in a low percentage and combined with another active, such as an avermectin. For example, U.S. Pat. No. 5,773,422 to Komer discloses ivermectin solutions with and without clorsulon. U.S. Pat. No. 8,362,086 to Soll et al., discloses long acting injectable formulations that may include an avermectin and up to about 10 percent (w/v) clorsulon. The combination used may target different types of parasites and may further be specific for a certain phase of a parasite's life cycle. Currently, antiparasitic products targeting liver flukes do not address the juvenile stage of the worm very well, requiring a combination strategy that may still not effectively address the problem of juvenile liver fluke resistance. In addition, the combination strategy brings with it the difficulties of coformulating, coadministering and accounting for the washout period of more than one active. The current antiparasitic strategy, accordingly, lacks in its efficacy a comprehensive treatment for all stages of liver fluke.

### INCORPORATION BY REFERENCE

Any abovementioned patents and published applications, and all documents cited therein or during their prosecution (“application cited documents”) and all documents cited or referenced in the application cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

Citation or identification of any document in this application is not an admission that such document is available as prior art to the present invention.

### SUMMARY OF THE INVENTION

The present invention is directed to compositions for treating helminth infestation comprising an anthelmintically effective amount of clorsulon as described herein and its use to control parasites in mammals. In accordance with this invention, it has been discovered that the composition shows unexpected efficacy for all life cycle stages of trematodes, including immature and adult liver flukes to include at least Fasciola hepatica, Fasciola gigantica and Fascioloides magna.

The invention encompasses uses or veterinary uses of injectable compositions comprising an anthelmintically effective amount of clorsulon for the treatment of parasitic trematode infections of animals (either wild or domesticated), including livestock and companion animals such as cats, dogs, horses, sheep, goats, pigs and cattle, with the aim of ridding these hosts of liver flukes encountered by such animals. The composition may also be suitable for humans.

The invention also provides methods for treating helminth infection comprising administration of an anthelmintically effective amount of clorsulon to an animal in need thereof. Surprisingly, it has been found that the inventive compositions and formulations described herein exhibit superior efficacy against F. hepatica compared to lower concentration clorsulon compositions known in the art. Certain embodiments of the invention include propylene carbonate and/or glycerol formal compositions which have the unexpected benefit of providing excellent syringeability at low temperatures. Certain embodiments also meet USP and Ph. Eur. requirements for antimicrobial effectiveness and accordingly do not require the addition of preservatives.

The invention does not intentionally seek to encompass any previously known product, process of making the product, or method of using the product such that the Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product. The invention and its embodiments are disclosed by the following Detailed Description.

### BRIEF DESCRIPTION OF THE DRAWINGS

FIG. 1 depicts the temperature dependent viscosity profiles of commercialized injectable solutions: Zactran® (diamond symbol), Ivomec® (square symbol), LongRange® (triangle symbol), and Ivomec® Gold (circle symbol); and 30% (w/v) clorsulon formulations in: glycerol formal (GF)/propylene glycol (PG) [42.0% w/v GF/QS to 100% PG] (“x” symbol), GF (asterisk symbol), and PC (‘+’).

FIG. 2 depicts the temperature dependent viscosity profiles of 30% (w/v) clorsulon formulations in: glycerol formal (GF)/propylene glycol (PG) [42.0% w/v GF/QS to 100% PG] (“x” symbol), GF (asterisk symbol), and propylene carbonate (‘+’).

FIG. 3 compares the syringibility (expressed as average force) of 30% (w/v) clorsulon in different solvents and solvent blends at 5° C. (left side bars) and 20° C. (right side bars) for each formulation. The solvents for each 5° C./20° C. temperature pair, reading left to right are: glycerol formal (“GF”); GF/PG (propylene glycol); GF/butyl acetate; propylene carbonate; and dimethyl isosorbide (“DMI”).

FIG. 4 depicts the temperature dependent viscosity profile of 30% (w/v) clorsulon in (a) 100% propylene carbonate (“PC”), “♦” diamond legend symbol, bottom plot, and in (b) 20% propylene glycol (“PG”) (w/v)/PC (qs to 100%), “x” legend symbol, top plot. The formulations (a) and (b) are listed in Table 1A, herein.

FIG. 5 depicts the temperature dependent viscosity profiles of 30% clorsulon in formulations listed from left to right in the legend as (a) 100/0, i.e. 100% propylene carbonate (“PC”) (diamond symbol), (b) 75/25 PC/glycerol formal (“GF”) (square symbol) (c) 50/50 PC/GF, (triangle symbol), (d) 30/70 PC/GF (‘x’ symbol), (e) 10/90 PC/GF (asterisk symbol), and (f) 0/100, i.e. 100% GF (‘+’ symbol). All ratios are in % w/v of the formulation.

FIG. 6 depicts the group mean clorsulon plasma levels in ng/mL for Study Number 5. Treatment groups (TRT) 2-5: TRT 5 is upper most curve, triangle symbol; TRT 4 is square symbol; TRT 3 is lower most curve, square symbol; TRT 2 is diamond symbol.

### DETAILED DESCRIPTION

In this disclosure and in the claims, terms such as “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

### Definitions

Terms used herein will have their customary meaning in the art unless specified otherwise.

As used herein, the term “about,” when referring to a value is meant to encompass variations of, in some embodiments±20%, in some embodiments±10%, in some embodiments±5%, in some embodiments±1%, in some embodiments±0.5%, and in some embodiments±0.1% from the specified amount, as such variations are appropriate to perform the disclosed methods or employ the disclosed compositions.

The expression “effective amount” as used herein means a concentration of the active agent in the composition sufficient to elicit the desired biological response to the target parasite(s) after administration of the composition to the animal, as measured by methods known in the art and/or described in the examples herein. In some embodiments, an “effective amount” of the active agent in the composition will provide an efficacy of at least 70% against the target parasite compared to an untreated control. In other embodiments, “an effective amount” of the active agent will preferably provide an efficacy of at least 80%, or at least 85% compared to untreated controls. In other embodiments, “an effective amount” of the active agent will provide an efficacy of at least 90%, at least 93%, at least 95% or at least 97% or at least 98% against the target parasite.

The abbreviation, “qs” or “QS” is used according to its customary meaning, namely, “quantum sufficit”. Thus, according to its customary meaning, qs to 100%, or qs 100% means to add a sufficient amount to equal 100%.

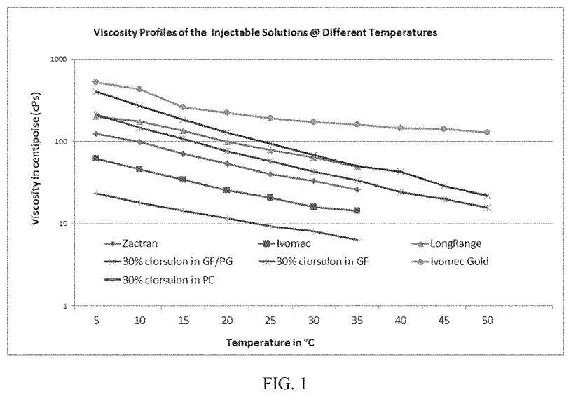

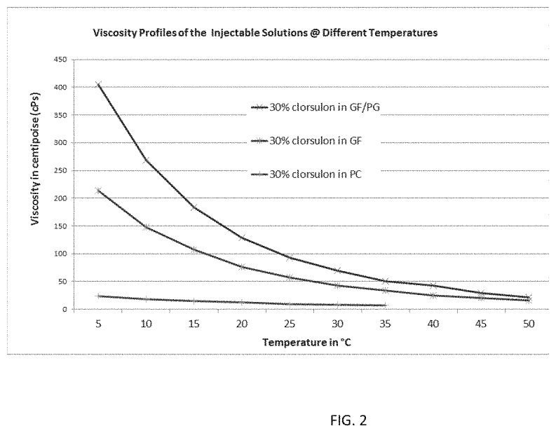

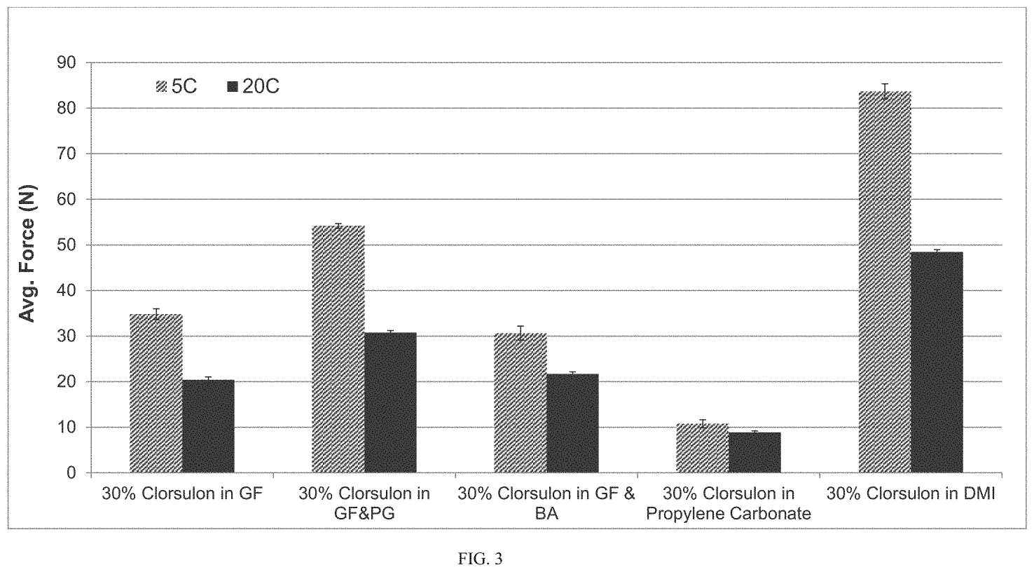

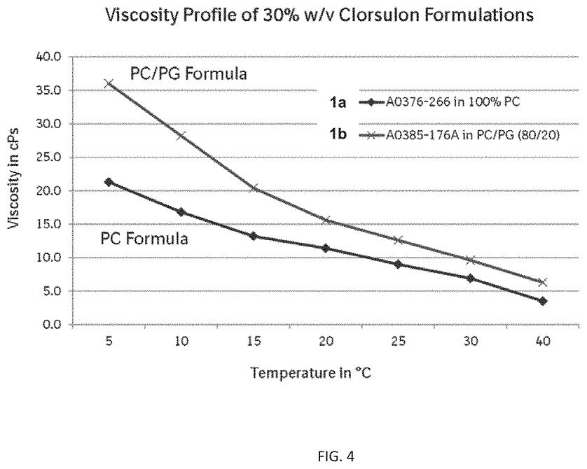

As referred to herein the term “injectable” in the context of fluids or liquids covers viscosities that are capable of being expelled by syringe and suitable for being administered to an animal via injection. FIG. 1, FIG. 2, and FIG. 4 depict the temperature dependent viscosity profiles of various injectable solutions. As used herein, a composition is suitable or appropriate for injection (i.e. has an acceptable syringeability) if it can be injected with a plunger force≤82 N. The plunger force may be measured using a laboratory experimental set-up to compare the syringeability of various solutions. One such set-up consists of a TA XT Plus Texture Analyzer (Texture Technologies) equipped with a cylindrical plate for pushing a syringe plunger, and a fixture to hold a syringe. Using this set-up, the plunger force to inject a composition at a rate (e.g. 3.3 mL/sec) through a needle (e.g. 16G×¾″) at a given temperature may be determined. By way of example, FIG. 3 depicts the injection force measured using the aforementioned set-up to evaluate 30% clorsulon (w/v) in different solvents and solvent blends at 5° C. and 20° C. As would be understood by one skilled in the art, some high viscosity compositions will exhibit acceptable syringeability at or below the specified plunger force if the composition exhibits shear thinning behavior (i.e. the apparent viscosity decreases with increased stress). Suitable viscosities for the invention are viscosities suitable at temperatures commonly encountered during actual use, such as about 5° C., 20° C., or temperatures therebetween. For certain compositions, a viscosity upper limit may be defined as acceptable for an injectable composition. For instance, viscosities at or below about 100 cP at 5° C. is suitable for injection of certain compositions, such as 30% (w/v) clorsulon compositions in propylene carbonate and/or glycerol formal.

Routes of injection may be parenteral, for example intramuscular (IM), intraperitoneal (IP), or subcutaneous (SQ). Another route of injection is intravenous, as an intravenous bolus. The locations of a subcutaneous injection may be various, as would be recognized by one skilled in the art to include the left or right side of the neck. The injection may be delivered anterior to or in front of the shoulder. Another injection location is the ear.

The injectable formulation may be packaged individually or in a multi-dose container. Multi-dose bottles or vials contain a volume of the formulation for dosing more than one animal. Multi-dose containers may be adapted to fit with an injector device, such as those sold commercially by Simcro™. A common dosage volume may be 5 mL, but lower or higher volumes may be used, ranging, for example from about 0.2 mL to about 20 mL. For example, subcutaneous injections might be limited to no more than about 20 mL at one injection site. About 10 mL is a common upper limit for intramuscular injection. Where larger volumes are needed, injection at multiple sites may be employed. Typically the volume will depend on the weight of the animal and the dose to be administered. Needle sizes may range from 14 G to 22 G, ½″ to 1.5″. For example, a common needle size that is useful for intramuscular injection of cattle, is 18 G×1 ½″. Other needle sizes commonly used for cattle, horses, hogs, pigs, and sheep are 16-gauge needles (16G). In smaller animals, such as dogs and cats, 20 G or 22 G needles may be used. For some cases, shorter needles, such as ½″ or 1 ″ needles are appropriate, especially for subcutaneous injections.

The term “animal” is used herein to include all mammals and also include all vertebrate animals. Animals include, but are not limited to, cats, dogs, cattle, cows, deer, goats, horses, llamas, pigs, sheep and yaks. It also includes an individual animal in all stages of development, including embryonic and fetal stages. In some embodiments, the animal may be a ruminant animal, such as beef cattle or dairy cattle.

In dairy cattle, for example, the cows alternate between “dry” and “lactating” periods. The dry period is, generally speaking, a rest and recovery time for the cow and the tissue comprising the udder before the calving period. The lactating period is, generally speaking, from calving to peak production levels at around 40 to 60 days after calving. The cow may then be bred. Production declines steadily afterwards, until, at about 305 days after calving, the cow is ‘dried off’ (i.e., prepared for the dry period), and milking ceases. About sixty days later, or about one year after the birth of her previous calf, a cow will calve again. Some cows are more difficult to breed at this approximate one-year interval; 13 or even 14 month cycles may more appropriate for these animals.

In a first aspect, the invention provides novel injectable veterinary compositions of clorsulon, as depicted in formula (I) below:

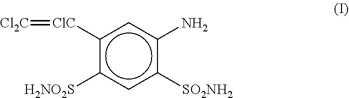

4-amino-6-(1,2,2-trichloroethenyl)benzene-1,3-disulfonamide (clorsulon)

According to one embodiment of the first aspect, the composition of the invention comprises about 25% (w/v) to about 35% (w/v) clorsulon in a solvent selected from glycerol formal, propylene carbonate, and mixtures thereof. The composition optionally includes a glycol solvent. In one embodiment, the glycol solvent is selected from propylene glycol, butylene glycol, and mixtures thereof, and optionally an antioxidant. In some embodiments, antioxidants such as an alpha tocopherol, ascorbic acid, ascrobyl palmitate, fumaric acid, malic acid, sodium ascorbate, sodium metabisulfate, n-propyl gallate, BHA (butylated hydroxy anisole), BHT (butylated hydroxy toluene) monothioglycerol and the like, may be added to the present formulation. In one embodiment, the antioxidant is butylated hydroxytoluene (BHT).

In some embodiments, the amount of BHT in the compositions according to the invention is about 0.005 to about 0.03%. In some embodiments, the amount of BHT is 0.01% (w/v) or 0.02% (w/v). In one embodiment, the amount of BHT is 0.02% (w/v).

In a preferred embodiment, the viscosity of the injectable composition according to the invention about 50 to about 150 cP at about 5° C. In a preferred embodiment the viscosity of the injectable composition according to the invention is less than or equal to about 125 cP at about 5° C. In another preferred embodiment, the viscosity of the injectable composition according the invention is about 80 to about 120 cP at about 5° C. In another preferred embodiment, the viscosity of the injectable composition according to the invention is less than about 100 cP at about 5° C.

The injectable compositions according to the invention preferably have efficacy against immature and adult stages of trematodes when the composition is administered as a single subcutaneous injection. In one embodiment, immature stages of trematodes are 4 week old trematodes, and adult stages of trematodes are older than 4 weeks. In certain embodiments, immature stages of trematodes are 2 weeks old or 3 weeks old. In certain embodiments, the compositions according to the invention show efficacy when administered as a single subcutaneous injection, and the efficacy against trematodes including Fasciola hepatica, is at least about 80% to 100%. In certain embodiments, the efficacy of a single subcutaneous injection dose of the compositions according to the invention against trematodes such as Fasciola hepatica is at least about 90%, 91%, 92%, 93%, 94%, 95%, 96%, 97%, 98%, or at least about 95%, or at least about 98%.

In certain embodiments, the injectable composition according to the invention is effective to kill at least about 80% of any 2, 3, and/or 4 week old trematodes infecting an animal when the composition is administered as a single subcutaneous injection. In certain embodiments, the injectable composition is effective to kill at least about 90% of any 2, 3, and/or 4 week old trematodes infecting an animal when the composition is administered as a single subcutaneous injection. In certain embodiments, the injectable composition is effective to kill at least about 92%, at least about 95%, or at least about 98% of any 2, 3, and/or 4 week old trematodes infecting an animal when the composition is administered as a single subcutaneous injection. In certain embodiments, the injectable composition is effective to kill at least about 80%, at least about 92%, at least about 95%, or at least about 98% of any 2, 3, and/or 4 week old Fasciola hepatica flukes infecting an animal when the composition is administered to the animal as a single subcutaneous injection.

In one embodiment, the injectable composition of the invention comprises glycerol formal. In some embodiments, the composition according to the invention comprises glycerol formal and propylene glycol. As known in the formulation art, glycerol formal is often used to solubilize water-insoluble compounds for aqueous dilution and is a chemical and dye emulsifier. Propylene glycol may be used as a solvent and/or as a stabilizing agent.

In one embodiment of the compositions of the invention, the amount of glycerol formal is about 20% (w/v) to about 60% (w/v). In one embodiment, the composition comprises about 20 to about 40% (w/v) clorsulon, about 20% to about 60% (w/v) glycerol formal, and propylene glycol, q.s. to 100% (w/v). In one embodiment, the composition comprises about 25 to about 35% (w/v) clorsulon, about 20% to about 60% (w/v) glycerol formal, and propylene glycol, q.s. to 100% (w/v).

In one embodiment of the compositions of the invention, the amount of glycerol formal is about 35% (w/v) to about 45% (w/v). In one embodiment, the composition comprises about 25 to about 35% (w/v) clorsulon, about 35% to about 45% (w/v) glycerol formal, and propylene glycol, q.s. to 100% (w/v).

In one embodiment, the composition consists essentially of about 25 to about 35% (w/v) clorsulon, about 35% to about 45% (w/v) glycerol formal, and propylene glycol, q.s. to 100% (w/v). In one embodiment, the composition according to the invention consists essentially of about 30% (w/v) clorsulon, about 42% glycerol formal (w/v), and propylene glycol, q.s. to 100% (w/v). In one embodiment, the composition according to the invention consists of about 30% (w/v) clorsulon, about 42% glycerol formal (w/v), and propylene glycol, q.s. to 100% (w/v). In one embodiment, the clorsulon, glycerol formal, propylene glycol compositions according to the invention also contain an antioxidant, such as BHT.

In one embodiment, the composition according to the invention comprises clorsulon, glycerol formal, and propylene carbonate. In one embodiment, the composition consists essentially of clorsulon, glycerol formal, and propylene carbonate. In one embodiment, the composition consists of clorsulon, glycerol formal, and propylene carbonate. In one embodiment, the clorsulon, glycerol formal, propylene carbonate compositions according to the invention also contain an antioxidant, such as BHT.

In certain embodiments of the invention, the weight ratio of propylene carbonate to glycerol formal is about 25:75 to about 95:5. In one preferred embodiment, the weight ratio of propylene carbonate to glycerol formal is about 30:70. In one embodiment of the invention, the amount of propylene carbonate is at least about 25% (w/v) or at least about 30% (w/v). In one embodiment, a composition according to the invention preferably consists essentially of about 30% w/v clorsulon, at least about 25% (w/v) or about 30% (w/v) propylene carbonate, and glycerol formal (q.s. to 100%). In one embodiment, a composition according to the invention preferably consists of about 30% w/v clorsulon, at least about 25% (w/v) or about 30% (w/v) propylene carbonate, and glycerol formal (q.s. to 100%). In certain embodiments, the clorsulon, propylene carbonate and glycerol formal compositions according to the invention also contain an antioxidant, such as BHT.

In one preferred embodiment, the injectable composition of the invention consists essentially of about 30% clorsulon (w/v), about 30% (w/v) propylene carbonate, and glycerol formal, q.s. to 100 mL. In one preferred embodiment, the invention consists of about 30% clorsulon (w/v), about 30% (w/v) propylene carbonate, and glycerol formal, q.s. to 100% (w/v).

In one embodiment, the invention is an injectable composition for treating helminth infection comprising about 25% (w/v) to about 35% (w/v) clorsulon in propylene carbonate (q.s. to 100% (w/v). In certain embodiments, the clorsulon/propylene carbonate compositions according to the invention included a glycol and/or an antioxidant, such as BHT. In certain embodiments, the clorsulon/propylene carbonate compositions according to the invention comprise propylene glycol, and optionally an antioxidant, such as BHT.

In one embodiment, the invention is an injectable composition consisting essentially of clorsulon, propylene carbonate, and propylene glycol. In certain embodiments, the clorsulon/propylene carbonate/propylene glycol compositions include an antioxidant, such as BHT. In one embodiment, the invention is an injectable composition consisting of clorsulon, propylene carbonate, and propylene glycol. In one embodiment, the invention is an injectable composition consisting of clorsulon, propylene carbonate, propylene glycol, and an antioxidant such as BHT. In a further embodiment of the invention, the amount of propylene glycol in the clorsulon/propylene carbonate/propylene glycol composition is about 15 to about 25% (w/v). In one embodiment, the amount of propylene glycol is about 20% (w/v). In one embodiment, the invention is an injectable composition consisting of about 30% (w/v) clorsulon, about 20% (w/v) propylene glycol, 0 to about 0.03% BHT, and propylene carbonate, q.s. to 100% (w/v).

According to one embodiment, an injectable composition of the invention consists essentially of clorsulon and propylene carbonate. According to one embodiment, the invention is an injectable composition consisting of clorsulon and propylene carbonate. According to one embodiment of the invention, the injectable compositions consist of clorsulon, propylene carbonate, and an antioxidant such as BHT. In one embodiment, the invention is an injectable composition consisting essentially of about 30% (w/v) clorsulon in propylene carbonate, q.s. to 100% (w/v). In one embodiment, the invention is an injectable composition consisting of about 30% (w/v) clorsulon, 0-0.03% BHT, and propylene carbonate (q.s. to 100% (w/v)).

### Methods of Treatment

In another aspect, the invention is a method for treating helminth infestation comprising administration of an anthelmintically effective amount of clorsulon to an animal in need thereof. In one embodiment, the concentration of clorsulon in the composition administered is about 25 to about 35% (w/v). In one embodiment, the helminths are trematodes. In another embodiment, the helminths are the liver fluke Fasciola hepatica, Fasciola gigantica or Fascioloides magna.

In one embodiment of the invention, methods for the treatment or prevention of a parasitic infection in a domestic animal are provided, which comprise administering an injectable composition comprising an effective amount of clorsulon active agent to the animal. The compositions and methods of the invention are effective against endoparasites, trematodes in particular, of animals and humans.

In one embodiment, the invention provides methods for the treatment and prevention of parasitic infections and infestations of animals (either wild or domesticated), including livestock and companion animals such as cats, dogs, horses, sheep, goats, pigs and cattle, with the aim of ridding these hosts of liver flukes commonly encountered by such animals.

By “treating” or “treat” or “treatment” is intended the application or administration of a composition of the invention to an animal that has a parasitic infestation for the eradication of the parasite or the reduction of the number of the parasites infesting the animal undergoing treatment. It is noted that the compositions of the invention may be used to prevent such a parasitic infestation.

In one embodiment, the invention is a method for treating helminth infection comprising the step of administering an effective amount of a composition of the first aspect as described herein to an animal in need thereof. In certain embodiments the composition is active against all stages of trematodes, including immature and adult trematodes. In one embodiment, the invention is a method of killing all stages of trematodes in an animal host comprising administering an effective amount of the composition of the first aspect to said animal. In certain embodiments, the invention provides a method of killing 4 week old, 3 week old, or 2 week old flukes in an animal host, wherein the method comprises administering an effective amount of the composition of the first aspect to said animal host.

In certain embodiments, the invention is a method for treating helminth infection comprising a step of administering a composition according to the first aspect to a ruminant animal, such as a bovine.

In one embodiment, the invention is a method for treating helminth infection comprising a step of administering a composition according to the first aspect to an animal in need thereof, wherein the step of administering the composition comprises a single subcutaneous injection at a dose of about 2-15 mg/kg (mass of clorsulon/kg animal body weight) or about 3-12 mg/kg; in certain embodiments, the dose is about 3, 4, 5, 6, 7, 8, 9, 10, 11 or 12 mg/kg.

In one embodiment, the invention is a method for treating helminth infection comprising a step of administering a composition according to the first aspect to an animal in need thereof, wherein the step of administering the composition comprises a single subcutaneous injection at a dose of about 4-8 mg/kg; in some embodiments, the dose is 4, 6, or 8 mg/kg (mass of clorsulon/kg animal body weight).

According to some embodiments of the invention, a method for treating helminth infection comprises the step of administering an effective amount of the composition of the first aspect to an animal in need thereof, wherein the helminths are trematodes selected from Fasciola hepatica, Fasciola gigantica, and Fascioloides magna. In a preferred embodiment, the invention is a method for treating helminth infection comprising a step of administering an effective amount of the composition of the first aspect to an animal in need thereof, wherein the helminth is Fasciola hepatica. In a preferred embodiment, the invention is a method for treating helminth infection comprising a step of administering an effective amount of the composition of the first aspect to a ruminant animal in need thereof, wherein the helminths is Fasciola hepatica. In a further preferred embodiment, the invention is a method for treating helminth infection comprising a step of administering an effective amount of a 30% (w/v) clorsulon and propylene carbonate composition of the first aspect to a ruminant animal in need thereof, wherein the helminths is Fasciola hepatica, and the composition consists essentially of about 30% (w/v) clorsulon in propylene carbonate.

In one embodiment, the invention provides a method of treatment wherein a single administration of a composition of the first aspect kills at least about 90% of any 2, 3 and/or 4 week old flukes infecting the animal. In another embodiment, a single administration of a composition of the first aspect kills at least about 90% of any 3 week old flukes, and/or at least 90% of any flukes younger than 3 weeks. In another embodiment, a single administration of a composition of the first aspect kills at least about 80%, or at least about 90% of any 2 week old flukes. According to another embodiment, the invention is a method wherein a single administration of a composition of the first aspect kills at least about 95% of any 2, 3, or 4 week old flukes infecting the animal. In certain embodiments, the invention is a method wherein a single administration of a composition of the first aspect kills at least about 95% of any flukes less than 4 weeks, less than 3 weeks, or any 3 week old or 2 week old flukes infecting the animal.

In another embodiment, a single administration of a composition according to the first aspect kills at least about 90%, at least about 95%, at least about 96%, at least about 97%, or at least about 98% of any flukes infesting the animal. In certain embodiments, a single administration of a composition according to the first aspect kills triclabendazole resistant flukes.

In one embodiment a single administration of a composition according to the first aspect kills at least about 90%, at least about 95%, or at least about 96%, 97%, or 98% of triclabendazole resistant flukes.

In one embodiment of the invention, a method for treating a trematode infection in an animal in need thereof comprises the step of administering a composition according to the first aspect, wherein the step of administering the composition comprises a single subcutaneous injection at a dose of about 6 to 8 mg clorsulon/kg animal body weight, and the dose is effective to kill at least about 80% to about 90%, at least about 80% to about 95%, at least about 90 to at least about 95%, at least about 80%, at least about 90%, or at least about 95% of any 2, 3, or 4 week old trematodes infecting the animal. In certain embodiments, a single subcutaneous injection at a dose of about 6 to 8 mg clorsulon/kg body weight kills at least about 90-95%, or any integer percent therebetween, of flukes infecting the animal. In certain embodiments, administering a single subcutaneous injection of the composition of the invention at a dose of 6 to 8 mg/kg clorsulon/body weight provides at least about 96% efficacy against trematodes such as Fasciola hepatica. In other embodiments administration of the composition as a single subcutaneous injection at a dose of 6 or 8 mg/kg clorsulon/body weight provides at least about 98% efficacy against trematodes such as Fasciola hepatica.

In one embodiment of the invention, a method for treating a trematode infection in an animal in need thereof comprises the step of administering a composition according to the first aspect, wherein the step of administering the composition comprises a single subcutaneous injection at a dose of about 6 or about 8 mg clorsulon/kg animal body weight, and the dose is effective to kill at least about 80 to about 95%, at least about 80% to about 90%, at least about 80%, or at least about 90%, or at least about 95% of any 2, 3, or 4 week old trematodes infecting the animal. In certain embodiments, a single subcutaneous injection at a dose of about 6 or about 8 mg clorsulon/kg body weight kills at least about 90-95%, or any integer percent therebetween, of flukes infecting the animal. In certain embodiments, administering a single subcutaneous injection of the composition of the invention at a dose of about 6 or about 8 mg/kg clorsulon/body weight provides at least about 96% efficacy against trematodes such as Fasciola hepatica. In other embodiments administration of the composition as a single subcutaneous injection at a dose of about 6 or about 8 mg/kg clorsulon/body weight provides at least about 98% efficacy against trematodes such as Fasciola hepatica.

In one embodiment, the invention is a method for treating a trematode infection in an animal comprising the step of administering an effective amount of a composition comprising (a) about 25% (w/v) to about 35% (w/v) clorsulon; (b) a solvent selected from glycerol formal, propylene carbonate, and mixtures thereof; (c) optionally, a glycol selected from butylene glycol, propylene glycol, and mixtures thereof; and (d) optionally, BHT. According to a further embodiment, the step of administering the composition comprises a single subcutaneous injection at a dose of about 6 or 8 mg clorsulon/kg animal body weight.

In one embodiment, the invention is a method for treating a trematode infection comprising administering a single subcutaneous injection dose of a composition according to the first aspect, wherein the dose is 6 or 8 mg clorsulon/kg animal body weight, and the composition consists essentially of about 30% w/v clorsulon and propylene carbonate.

In one embodiment, the invention is a method for treating a trematode infection comprising administering a single subcutaneous injection dose of a composition according to the first aspect, wherein the dose is 6 or 8 mg clorsulon/kg animal body weight, and the composition consists essentially of about 30% w/v clorsulon, propylene carbonate, and propylene glycol; in a further embodiment, the amount of propylene glycol is about 20% w/v.

In one embodiment, the invention is a method for treating a trematode infection comprising administering a single subcutaneous injection dose of a composition according to the first aspect, wherein the dose is 6 or 8 mg clorsulon/kg animal body weight, and the composition consists essentially of about 30% w/v clorsulon, propylene carbonate, and glycerol formal; in a further embodiment, the weight ratio of propylene carbonate to glycerol formal is 25:75 or higher.

In one embodiment, the invention is a method for treating trematode infection as described herein, wherein the trematodes comprise triclabendazole resistant trematodes. In another embodiment, the invention is a method for treating trematode infection as described herein, wherein the step of administering the composition comprises a single subcutaneous injection of a composition according to the first aspect at a dose that is effective to kill at least about 80%, at least about 90%, at least about 92%, at least about 94%, at least about 95%, or at least about 96%, 97%, or 98% of any triclabendazole resistant trematodes infecting the animal.

In some embodiments, the invention is a method of treating a trematode infection in an animal comprising the step of administering a composition according to first aspect in a volume that is about 0.5 mL, about 1.00 mL, about 1.5 mL, or about 2.0 mL per 50 kg body weight (“bwt”). In one embodiment, the composition according to first aspect of the invention is administered in a volume that is about 1.00 mL to about 1.33 mL/50 kg bwt.

### Additional Active Agents

In another aspect, the invention is the injectable clorsulon composition above with an additional active agent. Additional veterinary/pharmaceutical active ingredients may be used in accordance with all embodiments and aspects detailed above. In some embodiments, the additional active agents may include, but are not limited to, acaricides, anthelmintics, antiparasitics and insecticides. Anti-parasitic agents can include both ectoparasiticidal and endoparasiticidal agents.

In one embodiment, the additional active agent may be a macrocyclic lactone, such as abamectin, dimadectin, doramectin, emamectin, eprinomectin, ivermectin, latidectin, lepimectin, moxidectin, or selamectin. The additional active agent may be verapamil or another active agent discussed herein. Helminth infestation includes the presence of early immature to adult liver flukes to include Fasciola hepatica, Fasciola gigantica and Fascioloides magna.

In general, the additional active agent is included in the composition in an amount of between about 0.1 μg and about 1000 mg. More typically, the additional active agent may be included in an amount of about 10 μg to about 500 mg, about 1 mg to about 300 mg, about 10 mg to about 200 mg or about 10 mg to about 100 mg/mL.

In other embodiments of the invention, the additional active agent may be included in the composition to deliver a dose of about 5 μg/kg to about 50 mg/kg per weight of the animal. In other embodiments, the additional active agent may be present in an amount sufficient to deliver a dose of about 0.01 mg/kg to about 30 mg/kg, about 0.1 mg/kg to about 20 mg/kg, or about 0.1 mg/kg to about 10 mg/kg of weight of animal. In other embodiments, the additional active agent may be present in a dose of about 5 μg/kg to about 200 μg/kg or about 0.1 mg/kg to about 1 mg/kg of weight of animal. In still another embodiment of the invention, the additional active agent is included in a dose between about 0.5 mg/kg to about 50 mg/kg.

The compositions of the invention are made by mixing the appropriate amount of the active agents, pharmaceutically acceptable carrier or diluent and optionally a crystallization inhibitor, antioxidant, preservative, film former, etc., to form a composition of the invention. In some embodiments the composition can be obtained by following the method of making these forms described above by the description of making these forms found in general formulation text known to those in the art, e.g., Remington—The Science and Practice of Pharmacy (21^{st} Edition) (2005), Goodman & Gilman's The Pharmacological Basis of Therapeutics (11^{th} Edition) (2005) and Ansel's Pharmaceutical Dosage Forms and Drug Delivery Systems (8^{th} Edition), edited by Allen et al., Lippincott Williams & Wilkins, (2005).

The inventive formulations may contain other inert ingredients such as antioxidants, preservatives, or pH stabilizers. These compounds are well known in the formulation art. Antioxidants such as an alpha tocopherol, ascorbic acid, ascorbyl palmitate, fumaric acid, malic acid, sodium ascorbate, sodium metabisulfate, n-propyl gallate, BHA (butylated hydroxy anisole), BHT (butylated hydroxy toluene) monothioglycerol and the like, may be added to the present formulation. The antioxidants are generally added to the formulation in amounts of from about 0.01 to about 2.0%, based upon total weight of the formulation, such as about 0.05% to about 1.0%.

Preservatives, such as the parabens (methylparaben and/or propylparaben), are suitably used in the formulation in amounts ranging from about 0.01% to about 2.0%, or about 0.05% to about 1.0%. Other preservatives include benzalkonium chloride, benzethonium chloride, benzoic acid, benzyl alcohol, bronopol, butylparaben, cetrimide, chlorhexidine, chlorobutanol, chlorocresol, cresol, ethylparaben, imidurea, methylparaben, phenol, phenoxyethanol, phenylethyl alcohol, phenylmercuric acetate, phenylmercuric borate, phenylmercuric nitrate, potassium sorbate, sodium benzoate, sodium propionate, sorbic acid, thimerosal, and the like. Ranges for these compounds include from about 0.01% to about 5%.

Compounds which stabilize the pH of the formulation are also contemplated. Again, such compounds are well known to a practitioner in the art as well as how to use these compounds. Buffering systems include, for example, systems selected from the group consisting of acetic acid/acetate, malic acid/malate, citric acid/citrate, tartaric acid/tartrate, lactic acid/lactate, phosphoric acid/phosphate, glycine/glycimate, tris, glutamic acid/glutamates or sodium carbonate.

In addition to clorsulon, veterinary pharmaceutical agents that may be included in the compositions of the invention are well-known in the art (see e.g., Plumb' Veterinary Drug Handbook, 5th Edition, ed. Donald C. Plumb, Blackwell Publishing, (2005) or The Merck Veterinary Manual, 9^{th} Edition, (January 2005)) and include but are not limited to acarbose, acepromazine maleate, acetaminophen, acetazolamide, acetazolamide sodium, acetic acid, acetohydroxamic acid, acetylcysteine, acitretin, acyclovir, albendazole, albuterol sulfate, alfentanil, allopurinol, alprazolam, altrenogest, amantadine, amikacin sulfate, aminocaproic acid, aminopentamide hydrogen sulfate, aminophylline/theophylline, amiodarone, amitriptyline, amlodipine besylate, ammonium chloride, ammonium molybdenate, amoxicillin, clavulanate potassium, amphotericin B desoxycholate, amphotericin B lipid-based, ampicillin, amprolium, antacids (oral), antivenin, apomorphione, apramycin sulfate, ascorbic acid, asparaginase, aspiring, atenolol, atipamezole, atracurium besylate, atropine sulfate, aurnofin, aurothioglucose, azaperone, azathioprine, azithromycin, baclofen, barbituates, benazepril, betamethasone, bethanechol chloride, bisacodyl, bismuth sub salicylate, bleomycin sulfate, boldenone undecylenate, bromides, bromocriptine mesylate, budenoside, buprenorphine, buspirone, busulfan, butorphanol tartrate, cabergoline, calcitonin salmon, calcitrol, calcium salts, captopril, carbenicillin indanyl sodium, carbimazole, carboplatin, carnitine, carprofen, carvedilol, cefadroxil, cefazolin sodium, cefixime, cefoperazone sodium, cefotaxime sodium, cefotetan disodium, cefoxitin sodium, cefpodoxime proxetil, ceftazidime, ceftiofur sodium, ceftiofur, ceftiaxone sodium, cephalexin, cephalosporins, cephapirin, charcoal (activated), chlorambucil, chloramphenicol, chlordiazepoxide, chlordiazepoxide±clidinium bromide, chlorothiazide, chlorpheniramine maleate, chlorpromazine, chlorpropamide, chlortetracycline, chorionic gonadotropin (HCG), chromium, cimetidine, ciprofloxacin, cisapride, cisplatin, citrate salts, clarithromycin, clemastine fumarate, clenbuterol, clindamycin, clofazimine, clomipramine, claonazepam, clonidine, cloprostenol sodium, clorazepate dipotassium, clorsulon, cloxacillin, codeine phosphate, colchicine, corticotropin (ACTH), cosyntropin, cyclophosphamide, cyclosporine, cyproheptadine, cytarabine, dacarbazine, dactinomycin/actinomycin D, dalteparin sodium, danazol, dantrolene sodium, dapsone, decoquinate, deferoxamine mesylate, deracoxib, deslorelin acetate, desmopressin acetate, desoxycorticosterone pivalate, detomidine, dexamethasone, dexpanthenol, dexraazoxane, dextran, diazepam, diazoxide (oral), dichlorphenamide, diclofenac sodium, dicloxacillin, diethylcarbamazine citrate, diethylstilbestrol (DES), difloxacin, digoxin, dihydrotachysterol (DHT), diltiazem, dimenhydrinate, dimercaprol/BAL, dimethyl sulfoxide, dinoprost tromethamine, diphenylhydramine, disopyramide phosphate, dobutamine, docusate/DSS, dolasetron mesylate, domperidone, dopamine, doramectin, doxapram, doxepin, doxorubicin, doxycycline, edetate calcium disodium. calcium EDTA, edrophonium chloride, enalapril/enalaprilat, enoxaparin sodium, enrofloxacin, ephedrine sulfate, epinephrine, epoetin/erythropoietin, eprinomectin, epsiprantel, erythromycin, esmolol, estradiol cypionate, ethacrynic acid/ethacrynate sodium, ethanol (alcohol), etidronate sodium, etodolac, etomidate, euthanasia agents w/pentobarbital, famotidine, fatty acids (essential/omega), felbamate, fentanyl, ferrous sulfate, filgrastim, finasteride, fipronil, florfenicol, fluconazole, flucytosine, fludrocortisone acetate, flumazenil, flumethasone, flunixin meglumine, fluorouracil (5-FU), fluoxetine, fluticasone propionate, fluvoxamine maleate, fomepizole (4-MP), furazolidone, furosemide, gabapentin, gemcitabine, gentamicin sulfate, glimepiride, glipizide, glucagon, glucocorticoid agents, glucosamine/chondroitin sulfate, glutamine, glyburide, glycerine (oral), glycopyrrolate, gonadorelin, grisseofulvin, guaifenesin, halothane, hemoglobin glutamer-200 (OXYGLOBIN®), heparin, hetastarch, hyaluronate sodium, hydrazaline, hydrochlorothiazide, hydrocodone bitartrate, hydrocortisone, hydromorphone, hydroxyurea, hydroxyzine, ifosfamide, imidacloprid, imidocarb dipropinate, impenem-cilastatin sodium, imipramine, inamrinone lactate, insulin, interferon alfa-2a (human recombinant), iodide (sodium/potassium), ipecac (syrup), ipodate sodium, iron dextran, isoflurane, isoproterenol, isotretinoin, isoxsuprine, itraconazole, ivermectin, kaolin/pectin, ketamine, ketoconazole, ketoprofen, ketorolac tromethamine, lactulose, leuprolide, levamisole, levetiracetam, levothyroxine sodium, lidocaine, lincomycin, liothyronine sodium, lisinopril, lomustine (CCNU), lufenuron, lysine, magnesium, mannitol, marbofloxacin, mechlorethamine, meclizine, meclofenamic acid, medetomidine, medium chain triglycerides, medroxyprogesterone acetate, megestrol acetate, melarsomine, melatonin, meloxican, melphalan, meperidine, mercaptopurine, meropenem, metformin, methadone, methazolamide, methenamine mandelate/hippurate, methimazole, methionine, methocarbamol, methohexital sodium, methotrexate, methoxyflurane, methylene blue, methylphenidate, methylprednisolone, metoclopramide, metoprolol, metronidaxole, mexiletine, mibolerlone, midazolam milbemycin oxime, mineral oil, minocycline, misoprostol, mitotane, mitoxantrone, morphine sulfate, moxidectin, naloxone, mandrolone decanoate, naproxen, narcotic (opiate) agonist analgesics, neomycin sulfate, neostigmine, niacinamide, nitazoxanide, nitenpyram, nitrofurantoin, nitroglycerin, nitroprusside sodium, nizatidine, novobiocin sodium, nystatin, octreotide acetate, olsalazine sodium, omeprozole, ondansetron, opiate antidiarrheals, orbifloxacin, oxacillin sodium, oxazepam, oxibutynin chloride, oxymorphone, oxytretracycline, oxytocin, pamidronate disodium, pancreplipase, pancuronium bromide, paromomycin sulfate, parozetine, pencillamine, general information penicillins, penicillin G, penicillin V potassium, pentazocine, pentobarbital sodium, pentosan polysulfate sodium, pentoxifylline, pergolide mesylate, phenobarbital, phenoxybenzamine, pheylbutazone, phenylephrine, phenypropanolamine, phenytoin sodium, pheromones, parenteral phosphate, phytonadione/vitamin K-1, pimobendan, piperazine, pirlimycin, piroxicam, polysulfated glycosaminoglycan, ponazuril, potassium chloride, pralidoxime chloride, prazosin, prednisolone/prednisone, primidone, procainamide, procarbazine, prochlorperazine, propantheline bromide, propionibacterium acnes injection, propofol, propranolol, protamine sulfate, pseudoephedrine, psyllium hydrophilic mucilloid, pyridostigmine bromide, pyrilamine maleate, pyrimethamine, quinacrine, quinidine, ranitidine, rifampin, s-adenosyl-methionine (SAMe), saline/hyperosmotic laxative, selamectin, selegiline/l-deprenyl, sertraline, sevelamer, sevoflurane, silymarin/milk thistle, sodium bicarbonate, sodium polystyrene sulfonate, sodium stibogluconate, sodium sulfate, sodium thiosulfate, somatotropin, sotalol, spectinomycin, spironolactone, stanozolol, streptokinase, streptozocin, succimer, succinylcholine chloride, sucralfate, sufentanil citrate, sulfachlorpyridazine sodium, sulfadiazine/trimethroprim, sulfamethoxazole/trimethoprim, sulfadimentoxine, sulfadimethoxine/ormetoprim, sulfasalazine, taurine, tepoxaline, terbinafline, terbutaline sulfate, testosterone, tetracycline, thiacetarsamide sodium, thiamine, thioguanine, thiopental sodium, thiotepa, thyrotropin, tiamulin, ticarcilin disodium, tiletamine/zolazepam, tilmocsin, tiopronin, tobramycin sulfate, tocainide, tolazoline, telfenamic acid, topiramate, tramadol, trimcinolone acetonide, trientine, trilostane, trimepraxine tartrate w/prednisolone, tripelennamine, tylosin, urdosiol, valproic acid, vanadium, vancomycin, vasopressin, vecuronium bromide, verapamil, vinblastine sulfate, vincristine sulfate, vitamin E/selenium, warfarin sodium, xylazine, yohimbine, zafirlukast, zidovudine (AZT), zinc acetate/zinc sulfate, zonisamide and mixtures thereof.

In one embodiment of the invention, one or more macrocyclic lactones or lactams, which act as an acaricide, anthelmintic agent and/or insecticide, can be added to the compositions of the invention.

The macrocyclic lactones include, but are not limited to, avermectins, such as abamectin, dimadectin, doramectin, emamectin, eprinomectin, ivermectin, latidectin, lepimectin, selamectin, ML-1,694,554 and milbemycins, such as milbemectin, milbemycin D, moxidectin and nemadectin. Also included are the 5-oxo and 5-oxime derivatives of said avermectins and milbemycins. Examples of combinations of benzimidazole compounds with macrocyclic lactones include but may not be limited to those described in U.S. Pat. No. 7,396,820 (Virbac Corp. and Hartz Mountain Corporation), incorporated herein by reference. The '820 patent discloses a combination of fenbendazole with ivermectin along with at least two other active ingredients for the treatment of helminthiasis of mammals, and particularly tapeworm, hookworm, roundworm, whipworm and heartworm. The '820 patent does not contemplate the treatment of trematodes.

The macrocyclic lactone compounds are known in the art and can easily be obtained commercially or through synthesis techniques known in the art. Reference is made to the widely available technical and commercial literature. For avermectins, ivermectin and abamectin, reference may be made, for example, to the work “Ivermectin and Abamectin”, 1989, by M. H. Fischer and H. Mrozik, William C. Campbell, published by Springer Verlag., or Albers-Schönberg et al. (1981), “Avermectins Structure Determination”, J. Am. Chem. Soc., 103, 4216-4221. For doramectin, “Veterinary Parasitology”, vol. 49, No. 1, July 1993, 5-15 may be consulted. For milbemycins, reference may be made, inter alia, to Davies H. G. et al., 1986, “Avermectins and Milbemycins”, Nat. Prod. Rep., 3, 87-121, Mrozik H. et al., 1983, Synthesis of Milbemycins from Avermectins, Tetrahedron Lett., 24, 5333-5336, U.S. Pat. No. 4,134,973 and EP 0 677 054.

Macrocyclic lactones are either natural products or are semi-synthetic derivatives thereof. The structure of the avermectins and milbemycins are closely related, e.g., by sharing a complex 16-membered macrocyclic lactone ring. The natural product avermectins are disclosed in U.S. Pat. No. 4,310,519 and the 22,23-dihydro avermectin compounds are disclosed in U.S. Pat. No. 4,199,569. Mention is also made of U.S. Pat. Nos. 4,468,390, 5,824,653, EP 0 007 812 A1, U.K. Patent Specification 1 390 336, EP 0 002 916, and New Zealand Patent No. 237 086, inter alia. Naturally occurring milbemycins are described in U.S. Pat. No. 3,950,360 as well as in the various references cited in “The Merck Index” 12^{th} ed., S. Budavari, Ed., Merck & Co., Inc. Whitehouse Station, N.J. (1996). Latidectin is described in the “International Nonproprietary Names for Pharmaceutical Substances (INN)”, WHO Drug Information, vol. 17, no. 4, pp. 263- 286, (2003). Semisynthetic derivatives of these classes of compounds are well known in the art and are described, for example, in U.S. Pat. Nos. 5,077,308, 4,859,657, 4,963,582, 4,855,317, 4,871,719, 4,874,749, 4,427,663, 4,310,519, 4,199,569, 5,055,596, 4,973,711, 4,978,677, 4,920,148 and EP 0 667 054.

In another embodiment, the invention comprises a composition comprising clorsulon in combination with verapamil. Verapamil is believed to be an inhibitor of P-glycoprotein, which is a membrane protein that has been shown to efflux triclabendazole from triclabendazole-resistant F. hepatica. Inhibiting the efflux mechanism could allow the benzimidazole derivative to accumulate to toxic levels in the parasite.

In another embodiment, the invention comprises a composition comprising clorsulon in combination with a class of acaricides or insecticides known as insect growth regulators (IGRs). Compounds belonging to this group are well known to the practitioner and represent a wide range of different chemical classes. These compounds all act by interfering with the development or growth of the insect pests. Insect growth regulators are described, for example, in U.S. Pat. Nos. 3,748,356, 3,818,047, 4,225,598, 4,798,837, 4,751,225, EP 0 179 022 or U.K. 2 140 010 as well as U.S. Pat. Nos. 6,096,329 and 6,685,954 (all incorporated herein by reference).

In one embodiment the IGR is a compound that mimics juvenile hormone. Examples of juvenile hormone mimics include azadirachtin, diofenolan, fenoxycarb, hydroprene, kinoprene, methoprene, pyriproxyfen, tetrahydroazadirachtin and 4-chloro-2(2-chloro-2-methyl-propyl)-5-(6-iodo-3-pyridylmethoxy)pyridizine-3(2H)-one Examples of IGRs suitable for use include but are not limited to methoprene, pyriproxyfen, hydroprene, cyromazine, fluazuron, lufenuron, novaluron, pyrethroids, formamidines such as amitraz, 1-(2, 6-difluorobenzoyl)-3-(2-fluoro-4-(trifluoromethyl)phenylurea, and novaluron.

In one embodiment, the compositions of the invention comprise clorsulon in combination with methoprene and a pharmaceutically acceptable carrier.

In another embodiment, the IGR compound is a chitin synthesis inhibitor. Chitin synthesis inhibitors include chlorofluazuron, cyromazine, diflubenzuron, fluazuron, flucycloxuron, flufenoxuron, hexaflumoron, lufenuron, tebufenozide, teflubenzuron, triflumoron, 1-(2,6-difluorobenzoyl)-3-(2-fluoro-4-(trifluoromethyl)phenylurea, 1-(2,6-difluoro-benzoyl)-3-(2-fluoro-4-(1,1,2,2-tetrafluoroethoxy)-phenylurea and 1-(2,6-difluorobenzoyl)-3-(2-fluoro-4-trifluoromethyl)phenylurea.

In yet another embodiment of the invention, adulticide insecticides and acaricides can also be added to the composition of the invention. These include pyrethrins (which include cinerin I, cinerin II, jasmolin I, jasmolin II, pyrethrin I, pyrethrin II and mixtures thereof) and pyrethroids, and carbamates (which include but are not limited to benomyl, carbanolate, carbaryl, carbofuran, meththiocarb, metolcarb, promacyl, propoxur, aldicarb, butocarboxim, oxamyl, thiocarboxime and thiofanox).

In some embodiments, the compositions of the invention may include one or more antinematodal agents including, but not limited to, active agents in the benzimidazoles, imidazothiazoles, tetrahydropyrimidines, organophosphates class of compounds. In some embodiments, benzimidazoles including, but not limited to, thiabendazole, cambendazole, parbendazole, oxibendazole, mebendazole, flubendazole, fenbendazole, oxfendazole, albendazole, cyclobendazole, febantel, thiophanate and its o,o-dimethyl analogue may be included in the compositions.

In other embodiments, the compositions may include an imidazothiazole compounds including, but not limited to, tetramisole, levamisole and butamisole. In still other embodiments, the compositions of the invention may include tetrahydropyrimidine active agents including, but not limited to, pyrantel, oxantel, and morantel. Suitable organophosphate active agents include, but are not limited to, coumaphos, trichlorfon, haloxon, naftalofos and dichlorvos, heptenophos, mevinphos, monocrotophos, TEPP, and tetrachlorvinphos.

In other embodiments, the compositions may include the antinematodal compounds phenothiazine, piperazine as the neutral compound and in various salt forms, diethylcarbamazine, phenols such as disophenol, arsenicals such as arsenamide, ethanolamines such as bephenium, thenium closylate, and methyridine; cyanine dyes including pyrvinium chloride, pyrvinium pamoate and dithiazanine iodide; isothiocyanates including bitoscanate, suramin sodium, phthalofyne, and various natural products including, but not limited to, hygromycin B, α-santonin and kainic acid.

In other embodiments, the compositions of the invention may include other antitrematodal agents. Suitable antitrematodal agents include, but are not limited to, the miracils such as miracil D and mirasan; praziquantel, clonazepam and its 3-methyl derivative, oltipraz, lucanthone, hycanthone, oxamniquine, amoscanate, niridazole, nitroxynil, various bisphenol compounds known in the art including hexachlorophene, bithionol, bithionol sulfoxide and menichlopholan; various salicylanilide compounds including tribromsalan, oxyclozanide, clioxanide, rafoxanide, brotianide, bromoxanide and closantel; triclabendazole, diamfenetide, clorsulon, hetolin and emetine.

Anticestodal compounds may also be advantageously used in the compositions of the invention including, but not limited to, arecoline in various salt forms, bunamidine, niclosamide, nitroscanate, paromomycin and paromomycin II.

In yet other embodiments, the compositions of the invention may include other active agents that are effective against arthropod parasites. Suitable active agents include, but are not limited to, bromocyclen, chlordane, DDT, endosulfan, lindane, methoxychlor, toxaphene, bromophos, bromophos-ethyl, carbophenothion, chlorfenvinphos, chlorpyrifos, crotoxyphos, cythioate, diazinon, dichlorenthion, diemthoate, dioxathion, ethion, famphur, fenitrothion, fenthion, fospirate, iodofenphos, malathion, naled, phosalone, phosmet, phoxim, propetamphos, ronnel, stirofos, allethrin, cyhalothrin, cypermethrin, deltamethrin, fenvalerate, flucythrinate, permethrin, phenothrin, pyrethrins, resmethrin, benzyl benzoate, carbon disulfide, crotamiton, diflubenzuron, diphenylamine, disulfiram, isobornyl thiocyanato acetate, methoprene, monosulfiram, pirenonylbutoxide, rotenone, triphenyltin acetate, triphenyltin hydroxide, deet, dimethyl phthalate, and the compounds 1,5a,6,9,9a,9b-hexahydro-4a(4H)-dibenzofurancarboxaldehyde (MGK-11), 2-(2-ethylhexyl)-3a,4,7,7a-tetrahydro-4,7-methano-1H-isoindole-1,3(2H)dione (MGK-264), dipropyl-2,5-pyridinedicarboxylate (MGK-326) and 2-(octylthio)ethanol (MGK-874).

An antiparasitic agent that can be combined with the compounds of the invention to form a composition can be a biologically active peptide or protein including, but not limited to, depsipeptides, which act at the neuromuscular junction by stimulating presynaptic receptors belonging to the secretin receptor family resulting in the paralysis and death of parasites. In one embodiment of the depsipeptide, the depsipeptide is emodepside (see Willson et al., Parasitology, Jan. 2003, 126(Pt 1):79-86).

An insecticidal agent that can be combined with the compounds of the invention to form a composition can be a substituted pyridylmethyl derivative compound such as imidacloprid. Agents of this class are described above, and for example, in U.S. Pat. No. 4,742,060 or in EP 0 892 060. It would be well within the skill level of the practitioner to decide which individual compound can be used in the inventive formulation to treat a particular infection of an insect.

In certain embodiments, an insecticidal agent that can be combined with the compositions of the invention is a semicarbazone, such as metaflumizone.

In another embodiment, the compositions of the invention may advantageously include one or more isoxazoline compounds known in the art. These active agents are described in WO 2007/079162, WO 2007/075459 and US 2009/0133319, WO 2007/070606 and US 2009/0143410, WO 2009/003075, WO 2009/002809, WO 2009/024541, WO 2005/085216 and US 2007/0066617 and WO 2008/122375, all of which are incorporated herein by reference in their entirety.

In another embodiment of the invention, nodulisporic acid and its derivatives (a class of known acaricidal, anthelmintic, anti-parasitic and insecticidal agents) may be added to the compositions of the invention. These compounds are used to treat or prevent infections in humans and animals and are described, for example, in U.S. Pat. No. 5,399,582, 5,962,499, 6,221,894 and 6,399,786, all of which are hereby incorporated by reference in their entirety. The compositions may include one or more of the known nodulisporic acid derivatives in the art, including all stereoisomers, such as those described in the literature cited above.

In another embodiment, anthelmintic compounds of the amino acetonitrile class (AAD) of compounds such as monepantel (ZOLVIX) and the like may be added to the compositions of the invention. These compounds are described, for example, in WO 2004/024704; Sager et al., Veterinary Parasitology, 2009, 159, 49-54; Kaminsky et al., Nature vol. 452, 13 Mar. 2008, 176-181. The compositions of the invention may also include aryloazol-2-yl cyanoethylamino compounds such as those described in US 2008/0312272 to Soll et al., which is incorporated herein in its entirety, and thioamide derivatives of these compounds, as described in U.S. patent application Ser. No. 12/582,486, filed Oct. 20, 2009, which is incorporated herein by reference.

The compositions of the invention may also be combined with paraherquamide compounds and derivatives of these compounds, including derquantel (see Ostlind et al., Research in Veterinary Science, 1990, 48, 260-61; and Ostlind et al., Medical and Veterinary Entomology, 1997, 11, 407-408). The paraherquamide family of compounds are known class of compounds that include a spirodioxepino indole core with activity against certain parasites (see Tet. Lett. 1981, 22, 135; J. Antibiotics 1990, 43, 1380, and J. Antibiotics 1991, 44, 492). In addition, the structurally related marcfortine family of compounds, such as marcfortines A-C, are also known and may be combined with the formulations of the invention (see J. Chem. Soc.—Chem. Comm. 1980, 601 and Tet. Lett. 1981, 22, 1977). Further references to the paraherquamide derivatives can be found, for example, in WO 91/09961, WO 92/22555, WO 97/03988, WO 01/076370, WO 09/004432, U.S. Pat. Nos. 5,703,078 and 5,750,695, all of which are hereby incorporated by reference in their entirety.

Dosage forms may contain from about 0.5 mg to about 5 g of a combination of active agents, typically expressed as an amount per volume (w/v). In one embodiment of the dosage form, the amount of active is present in an amount of from about 1 mg to about 500 mg of an active agent, typically about 25 mg, about 50 mg, about 100 mg, about 200 mg, about 300 mg, about 400 mg, about 500 mg, about 600 mg, about 800 mg, or about 1000 mg. In one example, the volume of the amount of active may be 1 mL.

The compositions of the invention are administered in parasiticidally effective amounts which are which are suitable to control the parasite in question to the desired extent, as described below. In each aspect of the invention, the compounds and compositions of the invention can be applied against a single pest or combinations thereof.

The compositions of the invention may be administered continuously, for treatment or prevention of parasitic infections or infestations. In this manner, the compositions of the invention deliver an effective amount of the active compounds to the animal in need thereof to control the target parasites. By “effective amount” is intended a sufficient amount of a composition of the invention to eradicate or reduce the number of parasites infesting the animal. In some embodiments, an effective amount of the active agent achieves at least 70% efficacy against the target parasite. In other embodiments, an effective amount of the active agent achieves at least 80%, or at least 90% efficacy against the target pests. In other embodiments, an effective amount of the active agent will achieve at least 95%, at least 98% or 100% efficacy against the target parasites.

Generally, a dose of from about 0.001 to about 100 mg per kg of body weight given as a single dose or in divided doses for a period of from 1 to 5 days will be satisfactory but, of course, there can be instances where higher or lower dosage ranges are indicated, and such are within the scope of this invention. It is well within the routine skill of the practitioner to determine a particular dosing regimen for a specific host and parasite.

Higher amounts may be provided for very prolonged release in or on the body of the animal. In another treatment embodiment, the amount of active agents for animals which are small in size is greater than about 0.01 mg/kg, and in another embodiment for the treatment of small-sized animals the amount of active agents is between about 0.01 and about 20 mg/kg of weight of animal.

The solutions according to the invention may be applied using any means known per se, e.g., using an applicator gun or a metering flask, pipette, syringes, and other single dose and multi-dose containers.

In another aspect of the invention, a kit for the treatment or prevention of a parasitic infestation in an animal is provided, which comprises at least one isoxazoline active agent together with a pharmaceutically acceptable carrier and a dispensing device injectable application of the composition. The dispensing device may be single dose and multi-dose containers, which includes an effective dose of each active agent in the pharmaceutically acceptable carrier or diluent.

An important aspect of the invention is to provide a multiple-use container comprising an injectable composition of the invention, from which accurate single dose aliquots of the injectable formulations may be administered. The formulation must remain stable with repetitive exposure to the outside environment, particularly oxygen and water. This embodiment may be particularly useful with the very long lasting formulations of the invention that require administration to an animal infrequently, such as once every 3-6 months, or similar. Some solvents such as ethers (including DMI and the like) give rise to peroxides, which then yield ketones and aldehydes that may be further degraded to acids. The presence of acids may contribute to the degradation of acid hydrolysis-susceptible molecules, including isoxazoline active agents. Thus, formulation stability is particularly important for the multi-dose container application, where the formulations can be exposed to oxygen and water during multiple rounds of opening and closing. Importantly, it was found that the use of certain antioxidants such as BHT and BHA efficiently inhibit the degradation of the active agent in ether solvents.

Having thus described in detail various embodiments of the present invention, it is to be understood that the invention defined by the above paragraphs is not to be limited to particular details set forth in the above description as many apparent variations thereof are possible without departing from the spirit or scope of the present invention.

### Formulations

TABLE 1A
(temperature dependent viscosity profiles
of compositions depicted in FIG. 4)

| | Formula 1a | Formula 1b |
|---|---|---|
| | (30% ^{w}/ᵥ in PC) | (30% ^{w}/ᵥ in PC/PG) |
| Ingredient | % w/v | % w/v |
| Clorsulon USP | 30 | 30 |
| Propylene glycol | — | 20 |
| USP-NF, Ph. Eur., JP | | |
| Propylene carbonate USP-NF | QS | QS |

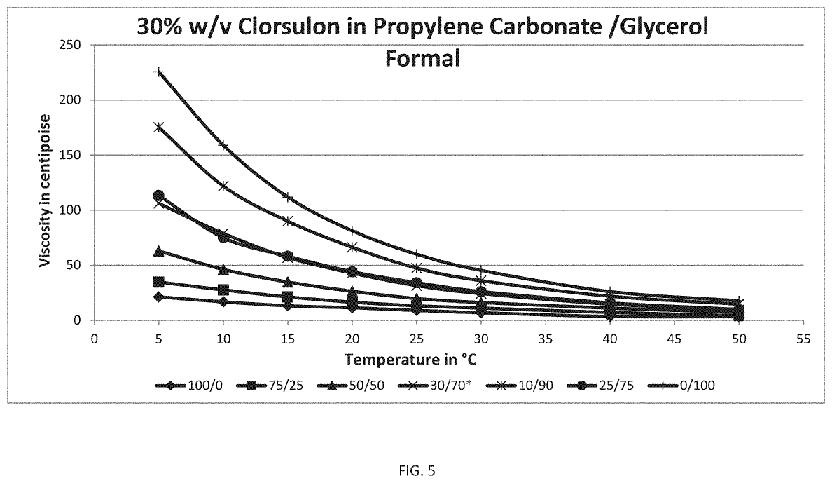

TABLE 1B
(temperature dependent viscosity profiles
of compositions depicted in FIG. 5)

| | Viscosity (cPs) of 30% w/v Clorsulon in | | | | | | |
|---|---|---|---|---|---|---|---|
| Temperature | Propylene Carbonate/Glycerol Formal¹ | | | | | | |
| (° C.) | 100/0 | 75/25 | 50/50 | 30/70² | 25/75 | 10/90 | 0/100 |
| 5 | 21.3 | 34.8 | 63 | 106.2 | 113.4 | 175.2 | 225.6 |
| 10 | 16.8 | 27.6 | 46.2 | 78.9 | 75 | 121.8 | 159 |
| 15 | 13.2 | 21.3 | 34.8 | 57 | 58.2 | 90.0 | 111.9 |
| 20 | 11.4 | 16.5 | 26.4 | 42.9 | 44.1 | 66.3 | 81.3 |
| 25 | 9 | 13.2 | 19.8 | 31.5 | 34.2 | 47.4 | 60 |
| 30 | 6.9 | 11.1 | 16.2 | 24.3 | 26.1 | 36.0 | 45.3 |
| 40 | 3.5 | 7.2 | 11.1 | 14.7 | 15.9 | 21.9 | 26.1 |
| 50 | 3.3 | 4.2 | 7.2 | 9.6 | 9.9 | 14.7 | 17.7 |

¹Viscosity analysis using LV-DV E Brookfield viscometer with S31 spindle, 100 rpm speed.
²30/70: 30% w/v of PC, then QS to 100% with glycerol formal (density calculated to be at 1.3079 g/mL).
Other formulas used PC solvent blends as noted with Glycerol Formal to QS.

TABLE 1C
Investigational Injectable Formulations tested in Studies 1-5

| Study Number | Investigational Formulations |
|---|---|
| Study 1 | 10% w/v clorsulon in 48.50% w/v Glycerol |
| | Formal (“GF”) plus Propylene Glycol |
| | (“PG”), QS to 100% (w/v) |
| | 30% w/v clorsulon in 42.0% w/v GF plus |
| | PG, QS to 100% (w/v). |
| Study 2 | 30% w/v clorsulon in 42.0% w/v GF plus |
| | PG, QS to 100% (w/v). |
| Study 3 | 30% w/v clorsulon in 42.0% w/v GF plus |
| | PG, QS to 100% (w/v). |
| Study 4 | Clorsulon 30% w/v in PC; (OS to 100% PC) |
| | Clorsulon 30% w/v in PC/BHT; (0.02% w/v |
| | BHT, QS to 100% w/v PC)” |
| | Clorsulon 30% w/v in PG/GF (42.0% w/v |
| | stabilized GF, QS to 100% w/v PC) |
| | Clorsulon 30% w/v in PG/PC (20.0% w/v |
| | PG, QS to 100% w/v PC) |
| Study 5 | Clorsulon 30% w/v in PC (QS to 100% PC) |

Notably, 30% (w/v) clorsulon in propylene carbonate and in propylene carbonate/propylene glycol (80/20) formulations as listed in Table 1A above showed favorable temperature-viscosity profiles (see FIG. 4) which also correlated with laboratory simulated acceptable injection force.

### Experimental Procedure and Results

### Study Number 1.

### Investigation of 10 and 30% (w/v) Clorsulon in Glycerol Formal/Propylene Glycol

Efficacy against liver flukes and plasma levels of clorsulon were evaluated following administration of 4, 6 or 8 mg/kg bodyweight as 10% w/v clorsulon or 30% w/v clorsulon by subcutaneous injection in cattle. The general study design was in accordance with the International Cooperation on Harmonisation of Technical Requirements for Registration of Veterinary Medicinal Products-VICH (International harmonisation of anthelmintic efficacy guidelines), “Efficacy of Anthelmintics: General Requirements” GL7 and “Efficacy of Anthelmintics: Specific Recommendations for Bovine” VICH GL12 (Vercruysse et al., 2001); and the “World Association for the Advancement of Veterinary Parasitology (W.A.A.V.P.) second edition of guidelines for evaluating the efficacy of anthelmintics in ruminants (bovine, ovine, caprine)” (Wood et al., 1995).

The study was a negative (untreated) controlled, blinded clinical efficacy and pharmacokinetic study using a randomized block design based on pre-treatment bodyweight as shown in Table 2.

TABLE 2

| | | | | | Total |
|---|---|---|---|---|---|
| Trt. | Investigational | | | Trt. | No. of |
| Group | Material | Dose | Route | Day | Animals |
| 1 | Untreated | NA | NA | NA | 5 |
| | Control | | | | |
| 2 | Clorsulon 30% | 1.33 mL/50 kg bwt | SC | Day | 5 |
| | w/v solution | (8 mg clorsulon/kg) | | 0 | |
| 3 | Clorsulon 30% | 1 mL/50 kg bwt | SC | Day | 5 |
| | w/v solution | (6 mg clorsulon/kg) | | 0 | |
| 4 | Clorsulon 30% | 1.33 mL/50 kg bwt | SC | Day | 5 |
| | w/v solution | (8 mg clorsulon/kg) | | 14 | |
| 5 | Clorsulon 30% | 1 mL/50 kg bwt | SC | Day | 5 |
| | w/v solution | (6 mg clorsulon/kg) | | 14 | |
| 6 | Clorsulon 30% | 1.33 mL/50 kg bwt | SC | Day | 5 |
| | w/v solution | (8 mg clorsulon/kg) | | 28 | |
| 7 | Clorsulon 30% | 0.67 mL/50 kg bwt | SC | Day | 5 |
| | w/v solution | (4 mg clorsulon/kg) | | 28 | |
| 8 | Clorsulon 10% | 2 mL/50 kg bwt | SC | Day | 5 |
| | w/v solution | (4 mg clorsulon/kg) | | 28 | |

Trt. = Treatment
SC = subcutaneously
bwt = bodyweight

The experimental unit was the individual cattle, which was identified, treated and assessed for the study variables on an individual basis.

Key steps in the schedule of operations for the study are organized in Table 3. Day 0 was not required to be the same calendar day for all animals but was the same calendar day for all animals in a block.

TABLE 3

| Study Day | Event |
|---|---|
| At least Day −35 | Study start (acclimation, health observations, etc.) |
| Day −28 | Inoculation of cattle with Fasciola hepatica |
| Day 0 | Treat Groups 2 and 3 |
| Day 14 | Treat Groups 4 and 5 |
| Day 28 | Treat Groups 6, 7 and 8 |
| Day 56 | Study conclusion (euthanasia and necropsy for |
| | all animals) |

TABLE 4
Test animal characteristics

| | |
|---|---|
| Species: | Bovine |
| Breed: | Not restricted, one breed/cross breed |
| | (specifically Deutsched |
| | Fleckvieh (German Simmental), |
| | Source Johann Langwieder, Seestr. |
| | 14, 83367 Petting, Germany). |
| Number: | 40 |
| Sex: | One sex (male) |
| Weight Range: | 135.0-200.5 kg |
| Age Range: | ≥3 months (actual 4-5 months) |
| Physiological Status: | Healthy (apart from fasciolosis), |
| | ruminating |

Animals were raised free of F. hepatica. Animals tested positive for Fasciola eggs by examination of fecal samples collected at least seven days prior to inoculation were not included in the study. Animals that have been treated with clorsulon within forty days prior to inoculation are debilitated, suffering from disease or injury, fractious or otherwise unsuitable for inclusion in the study, in the opinion of the Investigator, were excluded from the study. Any excluded animal received appropriate care.

After allocation, any animal suffering from disease or injury, or that became debilitated, fractious or otherwise unsuitable to remain in the study, in the opinion of the Investigator, was t removed.

Fasciola infection was induced by oral administration of infectious larval stages (metacercariae). Metacercariae was aliquoted from the same bulk and was administered on the same day. The inoculation schedule was designed so that F. hepatica were expected to be immature on Days 0, 14 and 28 (=treatment). A range of 400-500 infectious L3 larvae has produced moderate levels of infections and is generally in accord with the International Cooperation on Harmonization of Technical Requirements for Registration of Veterinary Medicinal Products” (VICH) GL7 (Food and Drug Administration (FDA) Guidance 90), Efficacy of anthelmintics: general requirements, and VICH GL12 (FDA Guidance 95), Efficacy of anthelmintics: specific recommendations for bovine (Vercruysse et al., Vet. Parasitol. 96 (2001) 171-193). The actual number of metacercariae given was recorded.

Personnel involved with collection of efficacy data were unaware as to treatment. Treatment assignments were not revealed until completion of all parasite counts for all blocks. Personnel performing parasite counts were given the samples in a nonsystematic way. In case of adverse events or human reaction, blinding codes were permitted to be revealed if deemed necessary.

Cattle were weighed once on the same day (Day-2±1) for allocation purpose and dose calculation (Groups 2 and 3). Weight was recorded, and the scale was verified before and after weighing of the animals. For dosing of Groups 4 to 8, respective animals were weighed the Day prior to treatment as per Schedule of Operations. The calculated dose was up to the next 0.2 mL increment, if it was not an exact 0.2 mL increment. Treatments were administered by subcutaneous injection using appropriately graduated disposable syringes.

Whole blood samples for analysis of plasma for analysis of clorsulon were collected from the jugular vein. Samples were collected in ˜7.5 mL lithium heparinized tubes for plasma preparation once prior to treatment on Day-2±1. Thereafter, blood samples were collected according to the Schedule of Operations (Table 3). The time of blood sampling was recorded, and care was taken to avoid cross-contamination of samples during sample collection, storage, and processing. Plasma was recovered from each sample, aliquoted (two aliquots) and stored frozen (at or below −20° C.) until required for assays. The cryo-tubes were labeled with Study Number, type of sample, date, Study Day/time of sampling, and animal ID.

One aliquot with plasma was used for analysis for clorsulon concentrations using currently High Performance Liquid Chromatography-Mass Spectrometry (LC-MS) methodology.

Fasciola hepatica counts were transformed to the natural logarithm of (count+1) for calculation of geometric means for each treatment group. Efficacy for each treated group was determined by calculating the percent efficacy as 100[(C-T)/C], where C is the geometric mean among untreated controls and T is the geometric mean among the treated animals.

Plasma samples were assayed for clorsulon. Data supporting the appropriate performance of the method during the bioanalytical phase of this study was included.

If quantifiable plasma concentrations were determined for an animal, pharmacokinetic analysis was performed as described below. Clorsulon plasma concentrations were determined for an animal at each sampling time, when appropriate. The area under the plasma concentration versus time curve from time 0 to the last quantifiable time point (AUC₀₋ₜ_{ₗₐₛₜ} was determined for an animal by the log-linear trapezoid method then averaged by treatment. The first order rate constant, λ_{z}, associated with the terminal log-linear portion of the curve was estimated via linear regression of the log plasma concentration versus time curve. The terminal plasma half-life was calculated using In (2)/ λ_{z}. The Cₘₐₓ and Tₘₐₓ for an animal were taken as the observed peak concentration and time to that observation. The half-life (t_{1/2}), Cₘₐₓ, and Tₘₐₓ were determined for an animal then averaged by treatment. The mean concentration-time profiles will be obtained by averaging the concentrations at each sampling time point for all treated animals.

At the study conclusion, worm counts were taken from the subjects' livers and gall bladders. The results of Study 1 are summarized in Table 5.

TABLE 5

| | | Animal | Fasciola hepatica, |
|---|---|---|---|
| Treatment Group | Block | ID | adult |
| 1 | 1 | 169 | 72 |
| Untreated Control | 2 | 443 | 72 |
| | 3 | 513 | 58 |
| | 4 | 454 | 129 |
| | 5 | 660 | 43 |
| | | GM | 69.0 |
| 2 | 1 | 315 | 1 |
| Clorsulon 30% w/v solution | 2 | 154 | 0 |
| at 1.33 mL/50 kg body weight | 3 | 382 | 2 |
| (8 mg/kg) subcutaneously | 4 | 318 | 10 |
| once on Day 0 | 5 | 408 | 0 |
| (four week old flukes) | | GM | 1.3 |
| | | Eff (%) | 98.1 |
| 3 | 1 | 798 | 0 |
| Clorsulon 30% w/v solution | 2 | 141 | 0 |
| at 1 mL/50 kg body weight | 3 | 128 | 0 |
| (6 mg/kg) subcutaneously | 4 | 767 | 2 |
| once on Day 0 | 5 | 325 | 2 |
| (four week old flukes) | | GM | 0.6 |
| | | Eff (%) | 99.1 |
| 4 | 1 | 209 | 0 |
| Clorsulon 30% w/v solution | 2 | 274 | 0 |
| at 1.33 mL/50 kg body weight | 3 | 079 | 0 |
| (8 mg/kg) subcutaneously | 4 | 910 | 0 |
| once on Day 14 | 5 | 412 | 0 |
| (six week old flukes) | | GM | 0 |
| | | Eff (%) | 100 |
| 5 | 1 | 102 | 0 |
| Clorsulon 30% w/v solution | 2 | 381 | 10 |
| at 1 mL/50 kg body weight | 3 | 735 | 0 |
| (6 mg/kg) subcutaneously | 4 | 504 | 0 |
| once on Day 14 | 5 | 591 | 0 |
| (six week old flukes) | | GM¹ | 0.6 |
| | | Eff² (%) | 99.1 |
| 6 | 1 | 669 | 1 |
| Clorsulon 30% w/v solution | 2 | 969 | 0 |
| at 1.33 mL/50 kg body weight | 3 | 467 | 0 |
| (8 mg/kg) subcutaneously | 4 | 406 | 0 |
| once on Day 28 | 5 | 599 | 0 |
| (eight week old flukes) | | GM | 0.1 |
| | | Eff (%) | 99.9 |
| 7 | 1 | 968 | 0 |
| Clorsulon 30% w/v solution | 2 | 456 | 0 |
| at 0.67 mL/50 kg body weight | 3 | 804 | 0 |
| (4 mg/kg) subcutaneously | 4 | 628 | 0 |
| once on Day 28 | 5 | 908 | 0 |
| (eight week old flukes) | | GM | 0 |
| | | Eff (%) | 100 |
| 8 | 1 | 741 | 0 |
| Clorsulon 10% w/v solution | 2 | 637 | 0 |
| at 2 mL/50 kg body weight | 3 | 327 | 0 |
| (4 mg/kg) subcutaneously | 4 | 247 | 1 |
| once on Day 28 | 5 | 583 | 0 |
| (eight week old flukes) | | GM | 0.1 |
| | | Eff (%) | 99.9 |

GM = Geometric mean parasite count
Eff = Efficacy (%) = 100 × (GM Control, Group 1 − GM Treated Group/GM Control, Group 1)

As shown in Table 5, the efficacy against early immature (four and six week old) F. hepatica of clorsulon administered subcutaneously to cattle at 6 and 8 mg/kg bodyweight as a 30% w/v solution was similar (>98-100%).

### Study Number 2.

### Investigation of 30% (w/v) Clorsulon in Glycerol Formal/Propylene Glycol

The therapeutic efficacy and pharmacokinetic parameters of clorsulon were evaluated when administered subcutaneously as 30% (w/v) clorsulon formulation to cattle experimentally infected with liver flukes (Fasciola hepatica). This study differs from Study Number 1 in that there is no administration of 10% (w/v) clorsulon and only immature (four week old) F. hepatica flukes were used.

The study was a negative (untreated) controlled, blinded clinical efficacy and pharmacokinetic study using a randomized block design based on pre-treatment bodyweight. The study design is shown in Table 6.

TABLE 6

| | | | | | Total |
|---|---|---|---|---|---|
| Trt. | Investigational | | | Trt. | No. of |
| Group | Material | Dose | Route | Day | Animals |
| 1 | Untreated | NA | NA | NA | 6 |
| | Control | | | | |
| 2 | Clorsulon 30% | 1.33 mL/50 kg bwt | SC | Day | 6 |
| | w/v solution | (8 mg clorsulon/kg) | | 0 | |
| 3 | Clorsulon 30% | 1 mL/50 kg bwt | SC | Day | 6 |
| | w/v solution | (6 mg clorsulon/kg) | | 0 | |
| 4 | Clorsulon 30% | 0.67 mL/50 kg bwt | SC | Day | 6 |
| | w/v solution | (4 mg clorsulon/kg) | | 0 | |

Trt. = Treatment
SC = subcutaneously anterior to the shoulder
No. = Number
bwt = bodyweight

Key steps in the schedule of operations for the study are organized in Table 7. Day 0 was not required to be the same calendar day for all animals but was the same calendar day for all animals in a block.

TABLE 7

| Study Day | Event |
|---|---|
| At least Day −35 | Study start (acclimation, health observations, etc.) |
| Day −28 | Inoculation of cattle with Fasciola hepatica |
| Day 0 | Treat Groups 2, 3 and 4 |
| Day 56 | Study conclusion (euthanasia and necropsy |
| | for all animals) |

Animal characteristics and exclusion and removal criteria were the same as in Study Number 1. The study model with respect to organism, parasite administration, blinding, parasite counts and data analysis were also the same as in Study Number 1.

At the study conclusion, worm counts were taken from the subjects' livers, gall bladders, and 50 cm of small intestine distal of the hepatic duct. The results of Study 2 are summarized in Table 8.

TABLE 8

| | | Animal | Fasciola hepatica, |
|---|---|---|---|
| Treatment Group | Block | ID | adult |
| 1 | 1 | 481 | 109 |
| Untreated Control | 2 | 365 | 147 |
| | 3 | 372 | 158 |
| | 4 | 240 | 140 |
| | 5 | 467 | 156 |
| | 6 | 163 | 140 |
| | | GM | 140.7 |
| 2 | 1 | 861 | 0 |
| Clorsulon 30% w/v solution | 2 | 998 | 1 |
| at 1.33 mL/50 kg body weight | 3 | 736 | 0 |
| (8 mg/kg) subcutaneously | 4 | 738 | 0 |
| once on Day 0 | 5 | 395 | 4 |
| (4 week old flukes) | 6 | 806 | 0 |
| | | GM | 0.5 |
| | | Eff (%) | 99.6 |
| 3 | 1 | 818 | 0 |
| Clorsulon 30% w/v solution | 2 | 963 | 1 |
| at 1 mL/50 kg body weight | 3 | 896 | 0 |
| (6 mg/kg) subcutaneously | 4 | 624 | 0 |
| once on Day 0 | 5 | 024 | 0 |
| (4 week old flukes) | 6 | 122 | 0 |
| | | GM | 0.1 |
| | | Eff (%) | 99.9 |
| 4 | 1 | 117 | 24 |
| Clorsulon 30% w/v solution | 2 | 488 | 41 |
| at 0.67 mL/50 kg body weight | 3 | 739 | 0 |
| (4 mg/kg) subcutaneously | 4 | 032 | 12 |
| once on Day 0 | 5 | 025 | 5 |
| (4 week old flukes) | 6 | 234 | 21 |
| | | GM | 10.0 |
| | | Eff (%) | 92.9 |

GM = Geometric mean parasite count
Eff = Efficacy (%) = 100 × (GM Control, Group 1 − GM Treated Group/GM Control, Group 1)

Parasite counts indicated efficacy of 99.6% (Group 2), 99.9% (Group 3), and 92.9% (Group 3) against 4 week old immature F. hepatica. All animals had systemic exposure to clorsulon. Maximum plasma concentrations were 3140±530 (Group 4), 4430±988 (Group 3) and 5740±1160 ng/mL (Group 2), and were reached within 12 hours (12 out of 18 animals).

### Study Number 3

### Investigation of 30% (w/v) Clorsulon in Glycerol Formal/Propylene Glycol

The therapeutic efficacy and plasma levels of clorsulon at 3, 6, or 12 mg/kg bodyweight administered subcutaneously as 30% clorsulon w/v solution against immature (three and four week old) F. hepatica was evaluated. The study was a negative (untreated) controlled, blinded clinical efficacy and pharmacokinetic study using a randomized block design based on pre-treatment bodyweight. The study design is shown in Table 9.

TABLE 9

| | | | | | Total |
|---|---|---|---|---|---|
| Trt.¹ | Investigational | | | Trt. | No.² of |
| Group | Material | Dose | Route | Day | Animals |
| 1 | Untreated | NA | NA | NA | 8 |
| | Control | | | | |
| 2 | Clorsulon 30% | 1 mL/50 kg bwt³ | SC⁴ | Day | 8 |
| | w/v solution | (6 mg clorsulon/kg) | | 0 | |
| 3 | Clorsulon 30% | 0.5 mL/50 kg bwt | SC | Day | 8 |
| | w/v solution | (3 mg clorsulon/kg) | | 7 | |
| 4 | Clorsulon 30% | 1 mL/50 kg bwt | SC | Day | 8 |
| | w/v solution | (6 mg clorsulon/kg) | | 7 | |
| 5 | Clorsulon 30% | 2 mL/50 kg bwt | SC | Day | 8 |
| | w/v solution | (12 mg clorsulon/kg) | | 7 | |

¹Trt. = Treatment
²No. = Number
³bwt = bodyweight
⁴SC = subcutaneous injection anterior to the shoulder

Animal characteristics and exclusion and removal criteria were the same as in Study Number 1. The study model with respect to organism, parasite administration, blinding, parasite counts and data analysis were the same as in Study Number 1.

At the study conclusion, worm counts were taken from the subjects' livers and gall bladders. The results of Study 3 are summarized in Table 10 (“GM” and “Eff” Notes are the same as in Study 1, Table 5).

TABLE 10
Individual and Group Mean Fasciola hepatica
Counts and % Efficacy

| | | Animal | Fasciola hepatica, |
|---|---|---|---|
| Treatment Group | Block | ID | adult |
| 1 | | 190 | 255 |
| Untreated Control | 2 | 731 | 246 |
| | 3 | 988 | 117 |
| | 4 | 301 | 234 |
| | 5 | 911 | 192 |
| | 6 | 910 | 123 |
| | 7 | 347 | 181 |
| | 8 | 678 | 72 |
| | | GM¹ | 164.3 |
| 2 | 1 | 797 | 23 |
| Clorsulon 30% w/v solution | 2 | 573 | 2 |
| at 1 ml/50 kg bodyweight | 3 | 446 | 16 |
| (6 mg/kg) subcutaneously | 4 | 230 | 0 |
| once on Day 0 | 5 | 906 | 72 |
| (3 week old flukes) | 6 | 974 | 0 |
| | 7 | 970 | 27 |
| | 8 | 599 | 5 |
| | | GM | 6.9 |
| | | Eff² (%) | 95.8 |
| 3 | 1 | 716 | 57 |
| Clorsulon 30% w/v solution | 2 | 610 | 64 |
| at 0.5 mL/50 kg bodyweight | 3 | 472 | 82 |
| (3 mg/kg) subcutaneously | 4 | 743 | 22 |
| once on Day 7 | 5 | 748 | 43 |
| (4 week old flukes) | 6 | 691 | 144 |
| | 7 | 357 | 17 |
| | 8 | 099 | 33 |
| | | GM | 47.0 |
| | | Eff (%) | 71.4 |
| 4 | 1 | 595 | 0 |
| Clorsulon 30% w/v solution | 2 | 652 | 2 |
| at 1 mL/50 kg bodyweight | 3 | 839 | 57 |
| (6 mg/kg) subcutaneously | 4 | 471 | 10 |
| once on Day 7 | 5 | 233 | 5 |
| (4 week old flukes) | 6 | 563 | 19 |
| | 7 | 548 | 14 |
| | 8 | 392 | 0 |
| | | GM¹ | 5.6 |
| | | Eff² (%) | 96.6 |
| 5 | 1 | 227 | 0 |
| Clorsulon 30% w/v solution | 2 | 953 | 0 |
| at 2 mL/50 kg bodyweight | 3 | 735 | 0 |
| (12 mg/kg) subcutaneously | 4 | 653 | 0 |
| once on Day 7 | 5 | 805 | 0 |
| (4 week old flukes) | 6 | 097 | 0 |
| | 7 | 348 | 7 |
| | 8 | 679 | 1 |
| | | GM | 0.4 |
| | | Eff (%) | 99.7 |

Based on geometric mean F. hepatica counts of untreated controls (Group 1) and Clorsulon 30% w/v solution-treated animals (Groups 2-5), percent efficacy was 95.8%, 71.4%, 96.8% and 99.7% for Groups 2, 3, 4 and 5, respectively, and Groups 2, 3, 4, and 5 animals had significantly fewer F. hepatica than the untreated controls (Group 1) (p<0.01 for all comparisons at α=0.10). All doses except the clorsulon 30% w/v at 3 mg/kg body weight were highly efficacious (>95%) against three and four week old F. hepatica. All animals accepted the treatment well and except of transient swelling of injection site observed in Group 2-5 animals, no health problems or adverse experience related to treatment occurred during the study.

Plasma concentrations in Groups 2-5 samples increased to a maximum concentration within one day followed by bi-exponential decay. The time to maximum concentration ranged from 0.333-0.5 days for Group 2, 0.165-0.5 days for Group 3, 0.5-1 day for Group 4, and 0.333-0.5 days for Group 5. The maximum concentrations (Cmax) were on average 6,640, 4,860, 7,320, and 15,700 ng/mL for Groups 2, 3, 4, and 5, respectively. The area under the curve from time 0 to the last sample time (AUClast) were on average 12,200, 6,870, 13,100, and 27,000 day*ng/mL for Groups 2, 3, 4, and 5, respectively. The average half-lives ranged from 4.48-5.62 days.

### Study Number 4

### Investigation of Various 30% Clorsulon Formulations

Efficacy and plasma levels of clorsulon when administered at 6 mg/kg bodyweight as different 30% (w/v) clorsulon formulations by subcutaneous injection to cattle. The study was a negative (untreated) controlled, blinded clinical efficacy and pharmacokinetic study using a randomized block design based on pre-treatment bodyweight. The study design is shown in Table 11.

TABLE 11

| | | | | | Total |
|---|---|---|---|---|---|
| Trt.¹ | Investigational | | | Trt. | No.³ of |
| Group | Material | Dose | Route² | Day | Animals |
| 1 | Untreated | NA | NA | NA | 8 |
| | Control | | | | |
| 2 | Clorsulon 30% | 1 mL/50 kg bwt⁴ | SC | Day | 8 |
| | w/v in PC | (6 mg clorsulon/kg) | | 0 | |
| 3 | Clorsulon 30% | 1 mL/50 kg bwt | SC | Day | 8 |
| | w/v in PC/BHT | (6 mg clorsulon/kg) | | 0 | |
| 4 | Clorsulon 30% | 1 mL/50 kg bwt | SC | Day | 8 |
| | w/v in PG/GF | (6 mg clorsulon/kg) | | 0 | |
| 5 | Clorsulon 30% | 1 mL/50 kg bwt⁴ | SC | Day | 8 |
| | w/v in PG/PC | (6 mg clorsulon/kg) | | 0 | |

¹Trt. = Treatment;
²SC = subcutaneously anterior to the shoulder;
³No. = Number;
⁴bwt = bodyweight

Animal characteristics, and the study model with respect to organism, parasite administration, blinding, parasite counts and data analysis were the same as in Study Number 1.

At the study conclusion, worm counts were taken from the subjects' livers, gall bladders, and 50 cm of small intestine distal of the hepatic duct. The results of Study 4 are summarized in Tables 12 and 13.

TABLE 12
Individual Fasciola hepatica Counts

| | | Animal | |
|---|---|---|---|
| Treatment Group | Block | ID | Fasciola hepatica |
| 1 | 1 | 4013 | 37 |
| Untreated Control | 2 | 1641 | 94 |
| | 3 | 3429 | 184 |
| | 4 | 7611 | 137 |
| | 5 | 1828 | 112 |
| | 6 | 3908 | 241 |
| | 7 | 3488 | 108 |
| | 8 | 9658 | 156 |
| 2 | 1 | 5816 | 10 |
| Clorsulon 30% w/v in | 2 | 0176 | 0 |
| Propylene carbonate at | 3 | 9494 | 0 |
| 1 mL/50 kg bodyweight | 4 | 8501 | 19 |
| (6 mg/kg) subcutaneously | 5 | 2054 | 14 |
| once on Day 0 | 6 | 4783 | 25 |
| (4 week old flukes) | 7 | 3419 | 0 |
| | 8 | 5733 | 0 |
| 3 | 1 | 9322 | 20 |
| Clorsulon 30% w/v in Propylene | 2 | 0454 | 1 |
| carbonate/Butylated | 3 | 1061 | 2 |
| hydroxytoluene | 4 | 4479 | 10 |
| at 1 mL/50 kg bodyweight | 5 | 4443 | 0 |
| (6 mg/kg) subcutaneously | 6 | 7188 | 0 |
| once on Day 0 | 7 | 5646 | 27 |
| (4 week old flukes) | 8 | 8040 | 20 |
| 4 | 1 | 5815 | 2 |
| Clorsulon 30% w/v in Propylene | 2 | 5732 | 6 |
| glycol/Glycerol formal | 3 | 8322 | 5 |
| at 1 mL/50 kg bodyweight | 4 | 7055A | 122 |
| (6 mg/kg) subcutaneously | 5 | 6758 | 6 |
| once on Day 0 | 6 | 6647 | 26 |
| (4 week old flukes) | 7 | 9181 | 2 |
| | 8 | 5158 | 2 |
| 5 | 1 | 2067 | 0 |
| Clorsulon 30% w/v in Propylene | 2 | 0463 | 0 |
| glycol/Propylene carbonate | 3 | 8245 | 8 |
| at 1 mL/50 kg bodyweight | 4 | 2806 | 10 |
| (6 mg/kg) subcutaneously | 5 | 2657 | 10 |
| once on Day 0 | 6 | 4870 | 2 |
| (4 week old flukes) | 7 | 1445 | 65 |
| | 8 | 0513 | 1 |

TABLE 13
Summary of Analysis Results of F. hepatica Counts
Counts for Control versus each Treated Group

| | Control | Treated | | |
|---|---|---|---|---|
| | Group | Group | % | |
| Comparison¹ | GM² | GM² | Efficacy³ | P-value⁴ |
| Group 1 versus 2 | 119.0 | 3.1 | 97.4 | <0.0001 |
| Group 1 versus 3 | 119.0 | 4.5 | 96.2 | <0.0001 |
| Group 1 versus 4 | 119.0 | 4.8 | 96.0 | <0.0001 |
| Group 1 versus 5 | 119.0 | 4.1 | 96.6 | <0.0001 |

¹Group 1 = Untreated control (n = 8 per group). Group 2 = Clorsulon 30% w/v in PC (n = 8 per group). Group 3 = Clorsulon 30% w/v in PC/BHT (n = 8 per group). Group 4 = Clorsulon 30% w/v in PG/GF (n = 7 per group). Group 5 = Clorsulon 30% w/v solution (n = 8 per group).
²GM = geometric mean.
³Percent efficacy = [(C − T)/C] × 100, where T and C are geometric means of each Treated Group and Control Group, respectively.
⁴P-value = two-sided probability value from analysis of variance on log-counts of each Treated Group and Control Group.

As shown above, all doses of clorsulon 30% w/v at 6 mg/kg body weight were highly efficacious (>96%) against four week old F. hepatica.

### Study Number 5

### Investigation of 30% (w/v) Clorsulon in Propylene Carbonate

Efficacy and plasma levels of clorsulon when administered at 3, 6, and 12 mg/kg bodyweight as 30% w/w clorsulon formulations by subcutaneous injection to cattle (once on day 0 or once on day 7).

TABLE 14
Total Individual Fasciola hepatica Counts and % Efficacy

| | | Animal | Fasciola hepatica, |
|---|---|---|---|
| Treatment Group | Block | ID | adult |
| 1 | 1 | 5716 | 83 |
| Untreated Control | 2 | 3902 | 119 |
| | 3 | 4899 | 162 |
| | 4 | 8013 | 211 |
| | 5 | 3311 | 137 |
| | 6 | 0481 | 58 |
| | 7 | 0311 | 83 |
| | 8 | 3400 | 117 |
| | | GM¹ | 112.8 |
| 2 | 1 | 6308 | 9 |
| Clorsulon 30% w/v injectable | 2 | 3569 | 27 |
| solution (in Propylene | 3 | 6751 | 6 |
| carbonate - “PC”) | 4 | 7254 | 4 |
| at 1 mL/50 kg | 5 | 5311 | 1 |
| bodyweight (equivalent to | 6 | 4901 | 1 |
| 6 mg clorsulon/kg bodyweight) | 7 | 5718 | 11 |
| once on Day 0. | 8 | 4898 | 7 |
| | | GM | 5.6 |
| | | Eff² (%) | 95.0 |
| 3 | 1 | 5719 | 3 |
| Clorsulon 30% w/v injectable | 2 | 3399 | 45 |
| solution (in PC) | 3 | 2676 | 18 |
| at 0.5 mL/50 kg | 4 | 3824 | 12 |
| bodyweight (equivalent to | 5 | 8102 | 50 |
| 3 mg clorsulon/kg bodyweight) | 6 | 3147 | 10 |
| once on Day 7. | 7 | 4093 | 145 |
| | 8 | 0381 | 7 |
| | | GM | 19.4 |
| | | Eff (%) | 82.8 |
| 4 | 1 | 9176 | 3 |
| Clorsulon 30% w/v injectable | 2 | 9294 | 1 |
| solution (in PC) | 3 | 2949 | 10 |
| at 1 mL/50 kg | 4 | 0383 | 2 |
| bodyweight (equivalent to | 5 | 7624 | 1 |
| 6 mg clorsulon/kg bodyweight) | 6 | 0108 | 0 |
| once on Day 7. | 7 | 4628 | 21 |
| | 8 | 8112 | 3 |
| | | GM | 2.8 |
| | | Eff (%) | 97.5 |
| 5 | 1 | 2302 | 0 |
| Clorsulon 30% w/v injectable | 2 | 1484 | 2 |
| solution (in PC) | 3 | 7101 | 0 |
| at 2 mL/50 kg | 4 | 1986 | 0 |
| bodyweight (equivalent to | 5 | 8586 | 0 |
| 12 mg clorsulon/kg bodyweight) | 6 | 3357 | 0 |
| once on Day 7. | 7 | 5075 | 0 |
| | 8 | 2677 | 0 |
| | | GM | 0.2 |
| | | Eff² (%) | 99.9 |

¹GM = Geometric mean parasite count
²Eff = Efficacy (%) = 100 × (GM Control, Group 1 − GM Treated Group/GM Control, Group 1)

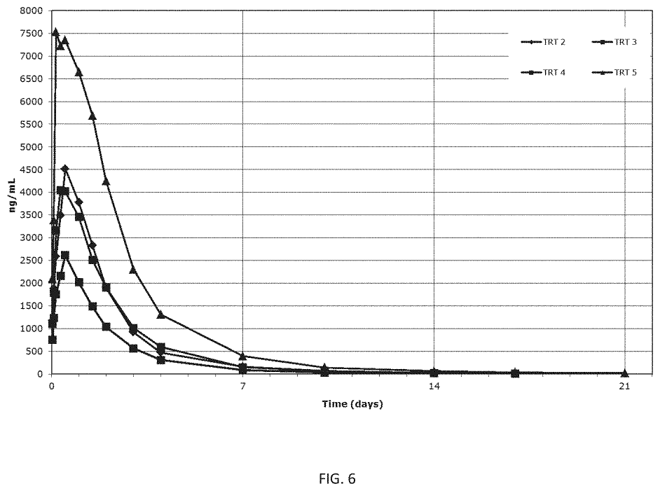

The group mean clorsulon plasma levels for this study (in ng/mL) are depicted in FIG. 6.

1. An injectable composition for treating helminth infection, comprising:
(a) about 25% (w/v) to about 35% (w/v) clorsulon;
(b) a solvent selected from glycerol formal, propylene carbonate, and mixtures thereof;
(c) optionally, a glycol; and
(d) optionally, BHT.

2. The composition of claim 1, wherein the solvent is glycerol formal.

3. The composition of claim 2, wherein the amount of glycerol formal is about 35% (w/v) to about 45% (w/v) of the composition.

4. The composition of claim 1, wherein the glycol is selected from butylene glycol, propylene glycol, and mixtures thereof

5. The composition of claim 1, wherein the solvent is propylene carbonate.

6. The composition of claim 5, wherein the composition consists essentially of clorsulon, propylene carbonate, and propylene glycol.

7. The composition of claim 6, wherein the amount of propylene glycol is about 15 to about 25% w/v.

8. The composition of claim 7 wherein the amount of propylene glycol is about 20% w/v.

9. The composition of claim 5, wherein the composition consists essentially of clorsulon and propylene carbonate.

10. The composition of claim 1, wherein the composition comprises glycerol formal and propylene carbonate.

11. The composition of claim 1, wherein the composition consists essentially of clorsulon, glycerol formal, and propylene carbonate.

12. The composition of claim 10 or 11, wherein the weight ratio of propylene carbonate to glycerol formal is about 25:75 to about 95:5.

13. The composition of claim 12, wherein the weight ratio of propylene carbonate to glycerol formal is about 30:70.

14. The composition of claim 13, wherein the amount of propylene carbonate is at least about 25% w/v.

15. The composition of claim 14, wherein the amount of propylene carbonate is at least about 30% w/v.

16. The composition of claim 1 further comprising a parasiticidally and/or pesticidally effective amount of an additional active agent.

17. The composition of claim 16, wherein the additional active agent is a macrocyclic lactone.

18. The composition of claim 17, wherein the macrocyclic lactone is abamectin, dimadectin, doramectin, emamectin, eprinomectin, ivermectin, latidectin, lepimectin, selamectin, or combinations thereof.

19. The composition of claim 16, wherein the additional active agent is verapamil.

20. The composition of claim 1, wherein the amount of clorsulon is about 30% w/v.

21. The composition of claim 1, wherein the composition is at least about 80% effective against immature and adult stages of trematodes when administered as a single subcutaneous injection

22. The composition of claim 1, wherein the composition is effective to kill at least about 90% of any 2, 3, and/or 4 week old trematodes infecting an animal when administered as a single subcutaneous injection.

23. The composition of claim 22, wherein the composition is effective to kill at least about 95% of any 2, 3, and/or 4 week old trematodes infecting an animal when administered as a single subcutaneous injection.

24. The composition of claim 23, wherein the composition is effective to kill at least about 98% of any 2, 3, and/or 4 week old trematodes infecting an animal when administered as a single subcutaneous injection.

25. A method for treating helminth infection comprising the step of administering an effective amount of the composition of claim 1 to an animal in need thereof.

26. The method of claim 25, wherein the step of administering the composition comprises a single subcutaneous injection of the composition at a dose of about 3, 4, 6, 8, 10, or 12 mg/kg clorsulon/animal body weight.

27. The method of claim 26, wherein the step of administering the composition comprises subcutaneous injection at a dose of about 4, 6, or about 8 mg/kg clorsulon/animal body weight.

28. The method of any one of claims 25-27, wherein the helminths are trematodes selected from the group consisting of Fasciola hepatica, Fasciola gigantica and Fascioloides magna.

29. A method of treating a trematode infection in an animal comprising the step of administering an effective amount of a composition comprising
(a) about 25% (w/v) to about 35% (w/v) clorsulon;
(b) a solvent selected from glycerol formal, propylene carbonate, and mixtures thereof;
(c) optionally, a glycol selected from butylene glycol, propylene glycol, and mixtures thereof; and
(d) optionally, BHT.

30. The method of claim 29, wherein the step of administering the composition comprises a single subcutaneous injection of the composition at a dose that is effective to kill at least about 80% of any 2, 3 and/or 4 week old trematodes infecting the animal.

31. The method of claim 30, wherein the step of administering the composition comprises a single subcutaneous injection of the composition at a dose that is effective to kill at least about 90% of any 2, 3 and/or 4 week old trematodes infecting the animal.

32. The method of claim 31, wherein the step of administering the composition comprises a single subcutaneous injection of the composition at a dose that is effective to kill at least about 95% of any 2, 3, and/or 4 week old trematodes infecting the animal.

33. The method of any one of claims 30-32, wherein the single subcutaneous injection is administered at a dose of about 6 or about 8 mg clorsulon/kg animal body weight.

34. The method of claim 33, wherein the composition consists essentially of about 30% w/v clorsulon and propylene carbonate.

35. The method of 33, wherein the composition consists essentially of about 30% w/v clorsulon, propylene carbonate, and propylene glycol.

36. The method of claim 35, wherein the amount of propylene glycol is about 20% w/v.

37. The method of claim 33, wherein the composition consists essentially of about 30% w/v clorsulon, propylene carbonate, and glycerol formal.

38. The method of claim 37, wherein the weight ratio of propylene carbonate relative to glycerol formal is about 25:75 or higher.

39. A method of treatment according to claims 25-38, wherein said trematodes comprise triclabendazole resistant trematodes.

40. An injectable composition or a method of treatment according to claim 1 or claim 29 wherein the viscosity of said composition is ≤100 cP at about 5° C.
